SEQUENCE LISTING (11 sequences)

SEQ ID NO: 1 - DNA, 96 bp, synthetic construct
  source 1..96: mol_type = other DNA
agctccagaagataaattacagggtagaggaagcgcgtctgccgcaagaccaacgaaacactaagataacgcacaactaggatactatgaccccgg

SEQ ID NO: 2 - DNA, 99 bp, synthetic construct
  source 1..99: mol_type = other DNA
agctccagaagataaattacagggaacgtgttggttgcggttcttccgatctgctgtgttctctatctgtgccatgcaactaggatactatgaccccgg

SEQ ID NO: 3 - DNA, 96 bp, synthetic construct
  source 1..96: mol_type = other DNA
agctccagaagataaattacaggatacaccaaggcatacgcaagcccctccaacgggcccatggccaggtcaccaactaggatactatgaccccgg

SEQ ID NO: 4 - DNA, 96 bp, synthetic construct
  source 1..96: mol_type = other DNA
agctccagaagataaattacagggacatcgtggccacttcatggggactgaattgaaccagatacgcctgctccaactaggatactatgaccccgg

SEQ ID NO: 5 - DNA, 96 bp, synthetic construct
  source 1..96: mol_type = other DNA
agctccagaagataaattacaggggccggggtggctcaggcaaggggttgacctgtcgtagggattgttttaacaactaggatactatgaccccgg

SEQ ID NO: 6 - DNA, 96 bp, synthetic construct
  source 1..96: mol_type = other DNA
agctccagaagataaattacaggcacggacaggccgcccgacccttcgaaacccacgatatgaaagcaacacccaactaggatactatgaccccgg

SEQ ID NO: 7 - DNA, 49 bp, synthetic construct
  source 1..49: mol_type = other DNA
gtaatagtcttcctgtccaagggttacttcgtcattaagatgtgttctg

SEQ ID NO: 8 - DNA, 49 bp, synthetic construct
  source 1..49: mol_type = other DNA
aaatttcgacccttaatatttcgctccgtccacgcgtactgattcgtgt

SEQ ID NO: 9 - DNA, 49 bp, synthetic construct
  source 1..49: mol_type = other DNA
atttctatgctgcagccaacttttccatacttccagcttaccatttatc

SEQ ID NO: 10 - RNA, 37 nt, synthetic construct
  source 1..37: mol_type = other RNA
tacgatccagtgggttgaaggaaagtaacagatcgta

SEQ ID NO: 11 - RNA, 47 nt, synthetic construct
  source 1..47: mol_type = other RNA
atatgatccagtgggttgaaggaaagtaacagatcgtatgagcgggg

(19) United States
(12) Patent Application Publication
(10) Pub. No.: US 2025/0011698 A1
(43) Pub. Date: Jan. 9, 2025

(54) COMPOSITIONS AND METHODS FOR REMOVAL OF HISTAMINE FROM FERMENTED LIQUIDS

(71) Applicant: Thomas Jefferson University, Philadelphia, PA (US)

(72) Inventor: Sangwon Kim, Philadelphia, PA (US)

(21) Appl. No.: 18/701,966

(22) PCT Filed: Oct. 18, 2022
(86) PCT No.: PCT/US2022/078309

§ 371 Date: Apr. 17, 2024

Related U.S. Application Data
(60) Provisional application No. 63/257,370, filed on Oct. 19, 2021.
(60) Provisional application No. 63/325,331, filed on Mar. 30, 2022.

(51) Int. Cl. C12H 1/056

(52) U.S. Cl. CPC C12H 1/0424

(57) ABSTRACT

The present disclosure relates, in part, to compositions and methods for the at least partial removal of histamine from a fermented liquid. In one aspect, composition of the present disclosure comprises a histamine binding domain conjugated to a solid support via a cross-linker. In certain embodiments, the histamine binding domain comprises an aptamer. In certain embodiments, the histamine binding domain comprises an anti-histamine antibody.

### CROSS-REFERENCE TO RELATED APPLICATIONS

This application claims priority under 35 U.S.C. § 119 (e) to U.S. Provisional Patent Application No. 63/325,331, filed Mar. 30, 2022, and U.S. Provisional Patent Application No. 63/257,370, filed Oct. 19, 2021, both of which applications are incorporated herein by reference in their entireties.

### SEQUENCE LISTING

The XML file named “205961-7072WO1_Sequence_Listing.xml” created on Oct. 7, 2022, comprising 10.9 Kbytes, is hereby incorporated by reference in its entirety.

### BACKGROUND

Histamine is a nitrogenous organic compound, endogenously produced in human mast cells, basophils, and/or enterochromaffin cells, which is involved in the regulation of a number of physiological functions, including but not limited to immune response and/or inflammation.

Stored endogenous histamine is released by immunogenic or hormonal stimuli (e.g., binding of an allergen to an IgE of a mast cell). However, dietary factors may also contribute to extracellular accumulation of histamine, as certain foods (e.g., meat and dairy) and beverages (e.g. fermented beverages) are known to contain histamine.

Elevated levels of extracellular histamine are associated with symptoms of the nasal mucosal membrane (e.g., runny nose, sneezing, itching, and/or congestion) and gastrointestinal tract (e.g., increased acidity) as a result of activation of histamine receptors (i.e., H1-H4). Extracellular histamine is ordinarily chemically degraded oxidatively via diamine oxidase (DAO) and intracellular histamine is degraded by methylation via histamine-N-methyltransferase (HNMT), thereby reducing histamine levels.

It is estimated that approximately 1% of the population suffers from histamine intolerance, which manifests as elevated levels of extracellular histamine upon exposure to exogenous histamine (e.g., consumption of wine, beer, sake, and/or kombucha, inter alia). Rapid accumulation of extracellular histamine in such individuals may occur due to reduced activity and/or production of degradation enzymes, such as DAO, resulting in an unpleasant experience upon consumption of exogenous histamine (e.g., drinking red wine). In addition, it has been shown that about 20% of the European population regularly take drugs that have DAO-inhibiting activity, increasing their susceptibility to histamine intolerance.

There is a need in the art for compositions and/or methods which partially or wholly remove histamine from fermented liquids. The present disclosure addresses this need.

### BRIEF SUMMARY

The present disclosure provides, in one aspect, a composition comprising a histamine binding domain conjugated to a solid support. In certain embodiments, the histamine binding domain is selected from the group consisting of an aptamer, a histamine binding fragment (Fab), a single-chain variable fragment (scFv), a single-domain antibody (sdAb), an anti-histamine antibody, and an anticalin. In certain embodiments, the histamine binding domain is an aptamer. In certain embodiments, the histamine binding domain is an anti-histamine antibody. In certain embodiments, the anti-histamine antibody is a polyclonal antibody.

In another aspect, the present disclosure provides a histamine scavenging apparatus comprising a porous container having a histamine-scavenging agent held therein, wherein the porous container comprises a plurality of pores having a diameter of sufficient size such that:
(a) passage of the histamine scavenging agent and optionally one or more particles within the liquid medium therethrough is prevented; and
(b) passage of a liquid medium therethrough is permitted.

In certain embodiments, the histamine scavenging agent comprises the histamine scavenging composition of the present disclosure (i.e., the composition comprising the histamine binding domain conjugated to a solid support).

In another aspect, the present disclosure provides a histamine scavenging apparatus comprising a column having a histamine scavenging agent held therein, an inlet for a liquid medium comprising histamine, and an outlet for a treated liquid medium with reduced histamine content, wherein the outlet comprises a porous and/or permeable material of sufficient porosity and/or permeability such that:
(a) passage of the histamine scavenging agent therethrough is prevented; and
(b) passage of the treated liquid medium therethrough is permitted.

In another aspect, the present disclosure provides a method of treating a fermented liquid comprising histamine, the method comprising contacting the fermented liquid with a histamine scavenging agent to provide a fermented liquid with reduced histamine content and a histamine adsorbed species.

In certain embodiments, the histamine scavenging agent comprises the histamine scavenging composition of the present disclosure (i.e., the composition comprising the histamine binding domain conjugated to a solid support).

In another aspect, the present disclosure provides a kit comprising the composition of the present disclosure, and instructional material for use thereof, wherein the instructional material comprises instructions for at least partially removing histamine from a fermented liquid.

In another aspect, the present disclosure provides a kit comprising a parcel containing the histamine scavenging composition of the present disclosure, a denaturant solution or low pH solution, a wash buffer solution, and a suspension buffer solution.

In another aspect, the present disclosure provides a method of at least partially removing histamine from a fermented liquid using the kit of the present disclosure to provide a fermented liquid with reduced histamine content, the method comprising:
contacting the parcel comprising the histamine scavenging composition with the fermented liquid to provide a fermented liquid with reduced histamine content and a parcel comprising a histamine adsorbed species;
separating the fermented liquid with reduced histamine content and the parcel comprising the histamine adsorbed species;
contacting the parcel comprising the histamine adsorbed species with the low pH or urea solution to provide a parcel comprising a first washed species;
contacting the parcel comprising the first washed species with the wash buffer solution to regenerate the parcel comprising the histamine scavenging composition; and
optionally storing the parcel comprising the histamine scavenging composition in the stored suspension buffer.

In certain embodiments, the fermented liquid is a fermented liquid beverage for human consumption. In certain embodiments, the beverage for human consumption is selected from the group consisting of wine, beer, cider, mead, sake, vinegar, soy sauce, tepache, and kombucha. In certain embodiments, the wine is red wine.

### BRIEF DESCRIPTION OF THE FIGURES

The drawings illustrate generally, by way of example, but not by way of limitation, various embodiments of the present application.

FIG. 1 provides an exemplary calibration curve for histamine in red grape juice.

FIG. 2 provides an exemplary chromatogram showing the retention time and concentration of histamine in a sample of red wine.

FIG. 3 provides exemplary extracted ion graphs generated for a sample of red wine.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “anticalin” as used herein refers to synthetic and/or artificial proteins which are able to bind to a specific antigen (e.g., histamine) with a high degree of specificity. Non-limiting examples of anticalins include lipocalins.

The term “aptamer” as used herein refers to single-stranded oligonucleotides (i.e., DNA or RNA) which fold into defined architectures which bind to a specific antigen (e.g., small molecules) with a high degree of specificity.

The term “antigen” as used herein is defined as a molecule that provokes an immune response (e.g., histamine). This immune response may involve either antibody production, or the activation of specific immunologically-competent cells, or both.

The terms “bead” as used herein refers to a morphology of a solid composition (e.g., a solid support) which may be generally characterized as having a rounded surface and/or roughly spherical shape. In certain embodiments the bead is porous. In certain embodiments, the bead is non-porous.

As used herein, the term “container” or “parcel” includes any receptacle for holding a composition. For example, in certain embodiments, the container is the packaging that contains the composition. In other embodiments, the container is not the packaging that contains the composition, i.e., the container is a receptacle, such as a box or vial that contains the packaged composition or unpackaged composition and the instructions for use of the composition. Moreover, packaging techniques are well known in the art. It should be understood that the instructions for use of the composition may be contained on the packaging containing the composition, and as such the instructions form an increased functional relationship to the packaged product. However, it should be understood that the instructions can contain information pertaining to the composition's ability to perform its intended function.

The term “cross-link” or “cross-linked” or “conjugated” as used herein refers to a covalent, non-covalent, or ionic bond that links one compound to another (e.g., a peptide and a nucleotide). In certain embodiments, a cross-link may refer to a conjugation of one compound to another with a direct bond between the two compositions. In yet other embodiments, cross-link may refer to a conjugation which relies on a cross-linker or cross-linking agent. A cross-linker is a chemical species which forms a covalent or ionic bond to each of two compounds at opposing termini of the cross-linker, thereby cross-linking the two compounds without a direct covalent or ionic bond between the two compounds.

As used herein “endogenous” refers to any material from or produced inside an organism, cell, tissue or system.

As used herein, the term “exogenous” refers to any material introduced from or produced outside an organism, cell, tissue or system.

The term “Fab” as used herein refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

The term “antigen binding domain” as used herein refers to any domain that binds to the antigen (e.g., histamine) and may include, but is not limited to, an aptamer, a monoclonal antibody, a polyclonal antibody, a synthetic antibody, a human antibody, a humanized antibody, a non-human antibody, single-domain antibody (sdAb), single-chain variable fragment (scFv), and an anticalin, and any fragment thereof. In some embodiments, the antigen binding domain portion comprises a mammalian antibody or a fragment thereof. In some embodiments, the antigen binding domain is selected from the group consisting of an aptamer, a variable region of an aptamer, an antibody, an antigen binding fragment (Fab), a variable region of single-domain antibody (sdAb), a single-chain variable fragment (scFv), and a variable region of an anticalin. In some embodiments, a histamine binding domain of the present invention is selected from the group consisting of a histamine specific aptamer, a histamine-specific antibody, a histamine-specific Fab, a histamine-specific single-domain antibody (sdAb), a histamine-specific scFv, and a histamine-specific anticalin. In one embodiment, a histamine binding domain is a histamine-specific antibody. In one embodiment, a histamine binding domain is a histamine-specific aptamer. In one embodiment, a histamine binding domain is a histamine-specific Fab. In one embodiment, a histamine binding domain is a histamine-specific scFv.

The term “histamine scavenging agent” as used herein refers to a composition and/or apparatus which is suitable to reduce an amount of histamine in a liquid medium in contact therewith. In certain embodiments, the histamine scavenging agent may selectively bind to and thereby sequester histamine in the liquid medium. In certain embodiments, a histamine binding domain may serve as a histamine scavenging agent.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “long term” as used herein refers to a period of time selected from the group consisting of one or more days, one or more weeks, and one or more months.

The term “reduced” as used herein in the context of histamine content and/or concentration, refers to a measurable decrease in the histamine content and/or concentration of a liquid medium (e.g., fermented liquid) after treatment with a histamine scavenging agent and/or histamine scavenging composition, as compared to the histamine content or concentration of the liquid medium before such treatment. In certain embodiments, the measurable decrease in histamine content and/or concentration is a decrease of more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%. In certain embodiments, reduction is such that the resultant liquid medium is substantially free of histamine.

The terms “sequence homology,” “percent identity (%),” “sequence identity,” “sequence identity percent,” or “percent identity” in the context of nucleotide and/or amino acid sequences refers to a quantitative measurement of the similarity between two sequences over an aligned region of the two sequences (e.g., two nucleotide sequences or two amino acid sequences).

The term “single-chain variable fragment” or “scFv” as used herein refers to a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker, which connects the N-terminus of the VH with the C-terminus of the VL, or the C-terminus of the VH with the N-terminus of the VL. In some embodiments, the antigen binding domain (e.g., histamine binding domain) comprises an scFv having the configuration from N-terminus to C-terminus, VH-linker-VL. In some embodiments, the antigen binding domain comprises an scFv having the configuration from N-terminus to C-terminus, VL-linker-VH. Those of skill in the art would be able to select the appropriate configuration for use in the present invention.

The term “solid support” as used herein refers to any inert, porous or non-porous solid substrate having a surface to which molecules may be attached, directly or indirectly, through either covalent or non-covalent bonds. The solid support may include any substrate material that is capable of providing physical support for the antigen binding domain(s) that are attached to the surface. The material is generally capable of enduring conditions related to the attachment of one or more antigen binding domains (e.g., aptamer oligonucleotides and/or antibodies) to the surface and any subsequent treatment, handling, or processing encountered during the use of compositions described herein. The materials which comprise the solid support may be naturally occurring, synthetic, or a modification of a naturally occurring material. Suitable solid support materials may include polysaccharides (e.g., sepharose and agarose, inter alia), silicon, graphite, mirrored surfaces, laminates, ceramics, and plastics (e.g., polyvinyl chloride, cyclo-olefin copolymers, polyacrylamide, polyacrylate, polyethylene, polypropylene, poly(4-methylbutene), polystyrene, polymethacrylate, poly(ethylene terephthalate), polytetrafluoroethylene (PTFE or Teflon®), steel, nylon, poly(vinyl butyrate)), either used by themselves or in conjunction with other materials. Additional solid support materials may be considered, non-limiting examples including glass (e.g., silica and/or bioglass). In certain embodiments, the solid support may comprise porous glass (e.g., controlled pore glass beads) and/or any other materials known in the art that are capable of having one or more functional groups suitable for covalent bond formation, non-limiting examples including an amino, carboxyl, thiol, and/or hydroxyl functional group. The solid support may adsorb a liquid phase, but is not completely dissolved therein. In certain embodiments, the solid support is magnetic. In certain embodiments, the porous or non-porous solid support is magnetic. The material used for a solid support may take any of a variety of configurations ranging from simple to complex. The solid support can have any one of a number of shapes, including a strip, plate, disk, rod, particle, including bead, tube, well, and the like. In certain embodiments, the solid support material is spherical (e.g., a bead), or cyclical (e.g., a column). The solid support material may be applied to the surface of another solid so as to provide a solid support coated object.

The term “single-domain antibody” or “sdAb” as used herein refers to an antibody fragment comprising a single monomeric variable antibody domain which is sufficient to selectively bind a specific antigen, without the light chain of the antibody.

The terms “specifically binds” or “selectively binds” as used herein with respect to an aptamer and/or antibody, means that the aptamer and/or antibody recognizes and/or binds to a specific antigen, but does not substantially recognize and/or bind to other molecules in a sample.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### DESCRIPTION

It is well known in the art that, in certain individuals, the consumption of food and/or beverages comprising histamine may result in allergic and/or gastrointestinal discomfort. Furthermore, certain food and/or beverages are more commonly associated with such issues than others, particularly fermented beverages (e.g., alcoholic or non-alcoholic beverages). Even among fermented beverages, the amount of histamine in each varies widely (Table 1). Red wine is most notable for its histamine content, which greatly exceeds many other fermented liquids, such as beer, white wine, and champagne (Biomolecules 2020, 10, 1181).

TABLE 1
Histamine content of common fermented beverages

| | Mean (SD) | Median | Minimum | Maximum |
|---|---|---|---|---|
| Fermented | Histamine | Histamine | Histamine | Histamine |
| beverage | (mg/kg) | (mg/kg) | (mg/kg) | (mg/kg) |
| White wine | 1.24 (1.69) | 0.45 | 0.10 | 13.00 |
| Red wine | 3.81 (3.51) | 1.90 | 0.09 | 55.00 |
| Beer | 1.23 (2.47) | 0.70 | — | 21.60 |

Accordingly, a number of products have been developed targeting the removal of histamine, as well as sulfites, from red wine, such as the PureWine wand (U.S. Pat. No. 9,670,442)

The present disclosure provides compositions and/or methods of selectively removing histamine from fermented liquids (e.g., red wine) without removal and/or degradation of sulfites and/or flavor-inducing chemical compounds. Treatment of the beverage for the removal of sulfites and/or flavor-inducing chemical compounds may lead to degradation and/or impact the flavor of a fermented beverage.

Thus, the histamine scavenging compositions and/or methods of the present disclosure may enable a subject to circumvent the symptoms associated with histamine intolerance upon consumption of a fermented beverage, without a compromise of the flavor of the fermented liquid from which the histamine is at least partially removed.

It is understood that, while the present disclosure describes the scavenging of histamine using the compositions, methods, and kits described herein, the disclosure is not limited to embodiments wherein the scavenged antigen is histamine. In certain embodiments, the antigen may include any of a number of chemical species, such as, for example, small molecules and/or polymeric species (e.g., organic polymers and/or resins, inorganic polymers, silica, polysiloxanes, polypeptides, proteins, oligonucleotides, and the like). In certain embodiments, the antigen is any of a number of small molecules and/or polymeric species present in a fermented beverage (e.g., wine), non-limiting examples including sulfites and tannins. Accordingly, the compositions, apparatuses, kits, and methods described herein may be adapted for use in scavenging alternative antigens.

### Compositions

In one aspect, the present disclosure provides a composition comprising a histamine binding domain conjugated to a solid support.

In certain embodiments, the histamine binding domain is selected from the group consisting of an aptamer, a histamine binding fragment (Fab), a single-chain variable fragment (scFv), a single-domain antibody (sdAb), an anti-histamine antibody, and an anticalin.

In certain embodiments, the histamine binding domain is an aptamer.

In certain embodiments, the aptamer comprises a DNA oligonucleotide. In certain embodiments, the DNA oligonucleotide shares at least 85%, 90%, 95%, 96%, 97%, 98%, or 99% sequence homology with A1 (SEQ ID NO:1). In certain embodiments, the DNA oligonucleotide shares at least 85%, 90%, 95%, 96%, 97%, 98%, or 99% sequence homology with A2 (SEQ ID NO: 2). In certain embodiments, the DNA oligonucleotide shares at least 85%, 90%, 95%, 96%, 97%, 98%, or 99% sequence homology with A3 (SEQ ID NO:3). In certain embodiments, the DNA oligonucleotide shares at least 85%, 90%, 95%, 96%, 97%, 98%, or 99% sequence homology with A4 (SEQ ID NO:4). In certain embodiments, the DNA oligonucleotide shares at least 85%, 90%, 95%, 96%, 97%, 98%, or 99% sequence homology with A5 (SEQ ID NO:5). In certain embodiments, the DNA oligonucleotide shares at least 85%, 90%, 95%, 96%, 97%, 98%, or 99% sequence homology with A6 (SEQ ID NO:6). In certain embodiments, the DNA oligonucleotide shares at least 85%, 90%, 95%, 96%, 97%, 98%, or 99% sequence homology with A7 (SEQ ID NO:7). In certain embodiments, the DNA oligonucleotide shares at least 85%, 90%, 95%, 96%, 97%, 98%, or 99% sequence homology with A8 (SEQ ID NO:8). In certain embodiments, the DNA oligonucleotide shares at least 85%, 90%, 95%, 96%, 97%, 98%, or 99% sequence homology with A9 (SEQ ID NO:9).

In certain embodiments, the DNA oligonucleotide is (SEQ ID NO:1). In certain embodiments, the DNA oligonucleotide is A2 (SEQ ID NO:2). In certain embodiments, the DNA oligonucleotide is A3 (SEQ ID NO:3). In certain embodiments, the DNA oligonucleotide is A4 (SEQ ID NO:4). In certain embodiments, the DNA oligonucleotide is A5 (SEQ ID NO:5). In certain embodiments, the DNA oligonucleotide is A6 (SEQ ID NO:6). In certain embodiments, the DNA oligonucleotide is A7 (SEQ ID NO:7). In certain embodiments, the DNA oligonucleotide is A8 (SEQ ID NO:8). In certain embodiments, the DNA oligonucleotide is A9 (SEQ ID NO:9).

In certain embodiments, the aptamer comprises a RNA oligonucleotide. In certain embodiments, the RNA oligonucleotide shares at least 85%, 90%, 95%, 96%, 97%, 98%, or 99% sequence homology with A10 (SEQ ID NO:10). In certain embodiments, the RNA oligonucleotide shares at least 85%, 90%, 95%, 96%, 97%, 98%, or 99% sequence homology with A11 (SEQ ID NO:11).

In certain embodiments, the RNA oligonucleotide is A10 (SEQ ID NO:10). In certain embodiments, the RNA oligonucleotide is A11 (SEQ ID NO:11).

In certain embodiments, the solid support is selected from the group consisting of controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, polyvinyl butyrate, agarose, and sepharose.

In certain embodiments, the solid support is a microparticle. In certain embodiments, the microparticle is magnetic. In certain embodiments, the solid support is a nanoparticle. In certain embodiments, the nanoparticle is magnetic. In certain embodiments, the microparticle is non-porous. In certain embodiments, the nanoparticle is porous. In certain embodiments, the microparticle is porous. In certain embodiments, the nanoparticle is non-porous.

In certain embodiments, the solid support has a spherical morphology. In certain embodiments, the solid support is a bead. In certain embodiments, the bead is a nanoparticle. In certain embodiments, the bead has a diameter selected from the group consisting of about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 nm. In certain embodiments, the bead has a diameter ranging from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nm. In certain embodiments, the bead is a microparticle. In certain embodiments, the bead has a diameter ranging from about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 μm. In certain embodiments, the bead is magnetic. In certain embodiments, the bead comprises steel. In certain embodiments, the bead is porous. In certain embodiments, the bead is non-porous.

In certain embodiments, the aptamer is conjugated to the solid support via at least one bond or bonding interaction selected from the group consisting of a covalent bond, an ionic bond, a hydrogen bond, and a Van der Waals bonding interaction.

In certain embodiments, the aptamer is conjugated to the solid support via a cross-linker. In certain embodiments, the cross-linker comprises polyethylenimine (PEI). In certain embodiments, the cross-linker comprises polyethylene glycol (PEG).

In certain embodiments, the histamine binding domain is an anti-histamine antibody.

In certain embodiments, the anti-histamine antibody is selected from the group consisting of a polyclonal antibody, a monoclonal antibody, a synthetic antibody, a human antibody, a humanized antibody, a non-human antibody, a single chain (scFv) antibody, a single-domain antibody (sdAb), or any fragment thereof.

In certain embodiments, the anti-histamine is a polyclonal antibody. In certain embodiments, the polyclonal antibody is produced in a rabbit. In certain embodiments, the polyclonal antibody is produced in a donkey. In certain embodiments, the polyclonal antibody is produced in a llama. In certain embodiments, the polyclonal antibody is produced in a camel. In certain embodiments, the polyclonal antibody is produced in a goat. In certain embodiments, the polyclonal antibody is produced in a sheep. In certain embodiments, the polyclonal antibody is produced in a horse. In certain embodiments, the polyclonal antibody is produced in a cow. In certain embodiments, the polyclonal antibody is produced in a mouse. In certain embodiments, the polyclonal antibody is produced in a rat. In certain embodiments, the polyclonal antibody is produced in a hamster. In certain embodiments, the polyclonal antibody is produced in a guinea pig. In certain embodiments, the polyclonal antibody is produced in a chicken.

In certain embodiments, the solid support is selected from the group consisting of sepharose, agarose, controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, and polyvinyl butyrate.

In certain embodiments, the solid support has a spherical morphology. In certain embodiments, the solid support is a bead. In certain embodiments, the bead is a nanoparticle. In certain embodiments, the bead has a diameter selected from the group consisting of about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or about 500 nm. In certain embodiments, the bead has a diameter ranging from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nm. In certain embodiments, the bead is a microparticle. In certain embodiments, the bead has a diameter ranging from about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or about 1000 μm. In certain embodiments, the bead is magnetic. In certain embodiments, the bead comprises steel. In certain embodiments, the bead is porous. In certain embodiments, the bead is non-porous.

In certain embodiments, the solid support is a microparticle. In certain embodiments, the microparticle is magnetic. In certain embodiments, the microparticle comprises steel. In certain embodiments, the solid support is a nanoparticle. In certain embodiments, the nanoparticle is magnetic. In certain embodiments, the nanoparticle comprises steel. In certain embodiments, the microparticle is non-porous. In certain embodiments, the nanoparticle is porous. In certain embodiments, the microparticle is porous. In certain embodiments, the nanoparticle is non-porous. In certain embodiments, the solid support comprises agarose. In certain embodiments, the solid support comprises sepharose. In certain embodiments, the solid support comprises Protein A sepharose. In certain embodiments, the solid support comprises Protein G sepharose. In certain embodiments, the Protein A sepharose has a particle size selected from the group consisting of about 40, 50, 60, 70, 80, 90, 100, 110, 120, 130, 140, 150, 160 and about 170 μm. In certain embodiments, the Protein A sepharose has an average particle size of about 90 μm. In certain embodiments, the protein A and histamine binding domain have a molar ratio of about 1:1, 1:2, 1:3, 1:4, 1:5, 1:6, 1:7, 1:8, 1:9, and about 1:10. In certain embodiments, the solid support is magnetic. In certain embodiments, the solid support is a magnetic particle. In certain embodiments, the magnetic particle is non-porous.

In certain embodiments, the bead comprises Protein G sepharose. In certain embodiments, the Protein A sepharose bead has a diameter selected from the group consisting of about 40, 50, 60, 70, 80, 90, 100, 110, 120, 130, 140, 150, 160 and about 170 μm. In certain embodiments, the Protein A sepharose bead has an average diameter of about 90 μm. In certain embodiments, the protein A and histamine binding domain have a molar ratio of about 1:1, 1:2, 1:3, 1:4, 1:5, 1:6, 1:7, 1:8, 1:9, and about 1:10. In certain embodiments, the magnetic particle is non-porous.

In certain embodiments, the composition of the present disclosure is packed in a column and/or funnel. In certain embodiments, contact of the composition of the present disclosure with a liquid medium provides a porous matrix. In certain embodiments, the porous matrix comprises a gel. In certain embodiments, the porous matrix is sufficiently buoyant as to float upon contact with the liquid medium. In certain embodiments, the porous matrix does not float upon contact with the liquid medium.

In certain embodiments, contact of composition of the present disclosure with a liquid medium provides a colloidal suspension. In certain embodiments, the colloidal suspension comprises granules of the composition of the present disclosure in the liquid medium. In certain embodiments, the granules are of sufficient buoyancy as to float upon contact with the liquid medium. In certain embodiments, the granules do not float upon contact with the liquid medium.

In certain embodiments, the histamine binding domain is conjugated to the solid support via a cross-linker. In certain embodiments, the cross-linker comprises suberic acid bis(N-hydroxysuccinimide).

### Aptamers

### Synthesis of Aptamers

In one aspect, the present disclosure provides a composition comprising a histamine binding domain conjugated to a solid support, wherein the histamine binding domain comprises a DNA or RNA aptamer, or chemically modified derivative thereof. The DNA or RNA aptamers, or chemically modified derivatives thereof, described herein may be prepared from commercially available starting materials, compounds known in the literature, or readily prepared intermediates, by employing standard synthetic methods and procedures known to those skilled in the art. Standard synthetic methods for the synthesis of polynucleotides and/or oligonucleotides are known to one skilled in the art, and can be readily obtained from the relevant scientific literature or from standard textbooks in the field.

Chemical synthesis of oligonucleotides can be accomplished using a number of protocols, including the use of solid support chemistry, wherein an oligonucleotide is synthesized one nucleoside at a time while anchored to an inorganic or organic polymer using a reactive group on the polymer, which reacts with a reactive group on the nucleoside to form a covalent linkage. Each subsequent nucleoside is then added to the first nucleoside molecule by: 1) formation of a phosphite linkage between the first nucleoside (i.e., nascent oligonucleotide) and a new nucleoside with a protecting group; 2) conversion of the phosphite linkage to a phosphate linkage by oxidation; and 3) removal of one of the protecting groups to form a new reactive site for the next nucleoside, as described in U.S. Pat. Nos. 4,458,066; 4,973,679; 5,132,418; 5,153,319; 5,554,744; and 6,441,159; all of which are incorporated herein by reference.

In certain embodiments, this procedure comprises anchoring the 3′-most nucleoside to a solid support functionalized with amine and/or hydroxyl moieties and subsequently adding the additional nucleosides in a stepwise fashion. Desired internucleotide linkages are formed between the 3′-functional group of the incoming nucleoside and the 5′-hydroxyl group of the 5′-most nucleoside of the nascent, support-bound oligonucleotide.

The process is generally initiated by coupling or “loading” of the 3′-most nucleoside of the desired oligonucleotide to a functionalized solid phase support or “resin”. A variety of solid supports and methods for their preparation are known in the art, non-limiting examples including, but not limited to, controlled pore glass (CPG), polystyrene, silica gel, cellulose paper, polyamide/kieselgur; polyacrylamide, and polacryloylmorpholide supports, as described in U.S. Pat. No. 7,777,023, which is incorporated herein by reference in its entirety, and in the literature (e.g., Curr. Protoc. Nucleic Acid Chem. 2013, Chapter 3, Unit 3.1; doi: 10.1002/0471142700.nc0301s53; and Current Protocols in Nucleic Acid Chemistry. 2000, 3.2.1-3.2.23).

In certain embodiments, the functionalized support comprises a plurality of functionalizable moieties (e.g., nucleophilic and/or electrophilic moieties suitable for covalent bond formation, including, but not limited to long chain alkyl amines or hydroxyl moieties) on the surface which serve as sites for nucleoside coupling.

In one non-limiting embodiment, DNA aptamers may be synthesized following classic methods for DNA oligonucleotide synthesis, such as the solid phase phosphoramidite process on an automated DNA synthesizer, for example, as described in Caruthers M H et al. (Methods Enzymol. 1987, 154:287-313) and U.S. Pat. Nos. 4,458,066; 4,973,679; 5,132,418; 5,153,319; 5,554,744; and 6,441,159; all of which are incorporated herein by reference.

RNA aptamers may be synthesized following any one of a number of synthetic methods, including solid phase synthetic methods, wherein said methods utilize varied immobilization (i.e., solid support linkage), protection/deprotection, and coupling steps, as described in the literature (e.g., Oligonucleotide Synth. 2005, 17-31; J. Am. Chem. Soc. 1987, 109:7845-7854; Nucleic Acids Res. 2007, 35:3287-3296; and Org. Lett. 2005, 7:3477-3480).

### Chemical Modifications of Aptamers

The aptamers described herein may comprise one or more chemical modifications which provide resistance to nuclease degradation or facilitate covalent and/or non-covalent linkage to a solid support.

Non-limiting examples of chemical modifications for increased resistance to nuclease degradation include, but are not limited to, terminal 3′-3′ or 5′-5′ inversion, 3′-biotin conjugation, 2′-substitutions, modifications of the phosphodiester linkage (e.g., methylphosphonate, phosphorothioate, and/or triazole linkages), and/or the use of non-natural nucleotides, as described in the literature (e.g., Int. J. Mol. Sci. 2017, 18:1683).

Nucleotides used in the synthesis of oligonucleotide aptamers can be modified in any of a number of ways, including modifications of the ribose and/or phosphate and/or base positions. Certain modifications are described in U.S. Pat. Nos. 5,660,985; 5,428,149; 5,580,972; 5,719,273, all of which are incorporated herein by reference. Additionally, oligonucleotide products may be modified subsequent to oligonucleotide synthesis, as described in U.S. Pat. No. 6,300,074.

### Identification of Suitable Aptamers

Methods for the screening and/or identifying aptamers that specifically bind to a target molecule or antigen (e.g., histamine) are known to those skilled in the art. A conventional method of aptamer selection is known as SELEX (Systematic Evolution of Ligands by Exponential Enrichment), as described in U.S. Pat. Nos. 5,475,096 and 5,270,163. SELEX allows the screening of oligonucleotides against a variety of target ligands (i.e., antigens) via an iterative and evolutionary process of continuous enrichment to identify target-specific binders. A typical SELEX library is vastly heterogenous with a large number of distinct nucleic acid molecules (approximately 10¹³ molecules). Each molecule folds into a unique secondary structure, which leads to a distinct geometrical shape. Depending on shape complementarity and non-covalent electrostatic or hydrophobic interactions, one or more DNA or RNA sequences can specifically bind to the desired target. Subsequently bound sequences are separated and amplified using Polymerase Chain Reaction (PCR) to generate an evolved library. The process is repeated until high-affinity binders are enriched, resulting in a homogenous library with high-affinity nucleic acid aptamers against the target of interest. SELEX has enabled the generation of a significant amount of aptamers against targets ranging from small molecules to whole cells.

Additional methods for the identification of aptamers with highly selective binding to a specific antigen include Ligand-Guided-Selection (LIGS), as described in U.S. Patent Appl. Pub. No. US 2019/0177716. Briefly, LIGS is a method for identifying highly specific aptamers against a predetermined antigen of a target. LIGS uses a highly specific binding (e.g., an antibody) interacting with its cognate antigen to displace specific aptamers from a partially enriched SELEX pool. Elution of the displaced aptamers provides aptamers that are specific to the predetermined antigen.

Utilizing the methods described herein for a particular antigen (e.g., histamine), a library of highly specific antigen binding aptamers may be prepared. Non-limiting examples of oligonucleotide sequences comprising aptamers of the present disclosure with specificity for histamine binding are described in Table 2.

TABLE 2
Selected aptamers with affinity for histamine binding

| | Nucleic | SEQ ID | |
|---|---|---|---|
| Aptamer | Acid | NO. | Nucleic Acid Sequence (5′ to 3′) |
| A1 | DNA | 1 | AGCTCCAGAAGATAAATTACAGGGTAGAGGAAGC |
| | | | GCGTCTGCCGCAAGACCAACGAAACACTAAGATA |
| | | | ACGCACAACTAGGATACTATGACCCCGG |
| A2 | DNA | 2 | AGCTCCAGAAGATAAATTACAGGGAACGTGTTGG |
| | | | TTGCGGTTCTTCCGATCTGCTGTGTTCTCTATCTGT |
| | | | GCCATGCAACTAGGATACTATGACCCCGG |
| A3 | DNA | 3 | AGCTCCAGAAGATAAATTACAGGATACACCAAGG |
| | | | CATACGCAAGCCCCTCCAACGGGCCCATGGCCAG |
| | | | GTCACCAACTAGGATACTATGACCCCGG |
| A4 | DNA | 4 | AGCTCCAGAAGATAAATTACAGGGACATCGTGGC |
| | | | CACTTCATGGGGACTGAATTGAACCAGATACGCC |
| | | | TGCTCCAACTAGGATACTATGACCCCGG |
| A5 | DNA | 5 | AGCTCCAGAAGATAAATTACAGGGGCCGGGGTG |
| | | | GCTCAGGCAAGGGGTTGACCTGTCGTAGGGATT |
| | | | GTTTTAACAACTAGGATACTATGACCCCGG |
| A6 | DNA | 6 | AGCTCCAGAAGATAAATTACAGGCACGGACAGG |
| | | | CCGCCCGACCCTTCGAAACCCACGATATGAAAG |
| | | | CAACACCCAACTAGGATACTATGACCCCGG |
| A7 | DNA | 7 | GTAATAGTCTTCCTGTCCAAGGGTTACTTCGTCA |
| | | | TTAAGATGTGTTCTG |
| A8 | DNA | 8 | AAATTTCGACCCTTAATATTTCGCTCCGTCCACG |
| | | | CGTACTGATTCGTGT |
| A9 | DNA | 9 | ATTTCTATGCTGCAGCCAACTTTTCCATACTTCC |
| | | | AGCTTACCATTTATC |
| A10 | RNA | 10 | UACGAUCCAGUGGGUUGAAGGAAAGUAACAGA |
| | | | UCGUA |
| A11 | RNA | 11 | AUAUGAUCCAGUGGGUUGAAGGAAAGUAACAG |
| | | | AUCGUAUGAGCGGGG |

### Conjugation of Aptamer(s) to a Solid Support

As described elsewhere herein, in certain embodiments, DNA and/or RNA oligonucleotide synthesis utilizes a solid support for iterative addition of nucleotides to a nascent oligonucleotide. Thus, it is appreciated that a solid support suitable for DNA and/or RNA oligonucleotide synthesis is contemplated for use as a solid support for the histamine scavenging agents described herein, wherein the histamine scavenging agent comprises an aptamer.

Non-limiting examples of solid supports suitable for use in the compositions described herein include controlled pore glass (CPG), silicon (e.g., SiO₂), graphite, mirrored surfaces, laminates, ceramics, and plastics (e.g., polyvinyl chloride, cyclo-olefine copolymers, polyacrylamide, polyacrylate, polyethylene, polypropylene, poly(4-methylbutene), polystyrene, polymethacrylate, poly(ethylene terephthalate), polytetrafluoroethylene (PTFE or Teflon®), steel, nylon, and poly(vinyl butyrate)), wherein the solid support is capable of having one or more functional groups suitable for covalent and/or non-covalent bond formation, non-limiting examples including amino, carboxyl, thiol, and/or hydroxyl functional groups, and wherein the solid support may possess any of a number of distinct shapes and/or morphologies (e.g., a bead).

In certain embodiments, the aptamer may be chemically modified with one or more functional groups suitable for covalent bond formation, such as a nucleophilic moiety (e.g., amine, hydroxyl, and/or thiol, inter alia), electrophilic moiety (e.g., alkyl halide, benzyl halide, allyl halide, propargyl halide, hydrazide, epoxide, amide, carboxyl, aldehyde, ester, and/or thioester, inter alia), diene or dienophile, and/or sp² hybridized halocarbon (e.g., aryl halide and/or vinyl halide). In certain embodiments, the solid support may comprise, or may be chemically modified so as to comprise, one or more functional groups suitable for covalent bond formation, such as a nucleophilic moiety (e.g., amine, hydroxyl, and/or thiol, inter alia), electrophilic moiety (e.g., alkyl halide, benzyl halide, allyl halide, propargyl halide, carboxyl, ester, and/or thioester, inter alia), diene or dienophile, and/or sp² hybridized halocarbon (e.g., aryl halide and/or vinyl halide). In certain embodiments, the functional groups suitable for covalent bond formation of the aptamer and the solid support are selected such that covalent bond formation (i.e., conjugation) may be achieved with contact of a solid support and aptamer having complementary functional groups, optionally in the presence of one or more additional reagents and/or catalysts, as appropriate to achieve the desired transformation. For example, conjugation may be achieved by contacting an aptamer comprising an amine (e.g., terminal 3′-substituted amine) and a solid support comprising a carboxyl or activated carboxylic acid derivative, in the presence of a suitable coupling reagent (e.g., 1-ethyl-3-(3-dimethylaminopropyl) carbodiimide (EDC)), a base (e.g., Et₃N), and/or a catalytic amount of acid (e.g., HCl).

Non-limiting examples of complementary functional groups for conjugation of an aptamer and solid support include: (a) a nucleophile and electrophile (e.g., SN₁ or SN₂ reaction of an hydroxyl and benzyl chloride or an amine and a carboxylic acid or derivative thereof); (b) an azide and an alkyne (i.e., [3+2] cycloaddition or “click” reaction); (c) a diene and a dienophile (e.g., substituted butadiene and substituted maleimide) via a Diels-Alder [4+2] cycloaddition.

It is appreciated that any of a number covalent bond forming reactions (e.g., SN₂, condensation, Diels-Alder reaction (i.e., [4+2] cycloaddition), [3+2] dipolar cycloaddition, and transition metal catalyzed cross-coupling, inter alia) may be employed to prepare the conjugated compositions of the present disclosure. It is understood that, given a particular bond forming reaction (e.g., SN₂ reaction), one skilled in the art would readily recognize the requisite functional groups suitable for each component (i.e., aptamer and solid support) necessary to achieve conjugation. Additionally, one skilled in the art of organic synthesis would be apprised of the necessary additional reagents and/or catalyst necessary to achieve covalent bond formation.

It is further appreciated that, in certain embodiments, chemical modification of one or more of the aptamer and solid support may not be necessary to achieve the complementary functionality required for covalent or non-covalent bond formation. For example, an unprotected oligonucleotide comprises at least two hydroxyl moieties and polystyrene beads comprising a benzyl chloride substituent are commercially available (i.e., Merrifield resin LL (100-200 mesh)).

In certain embodiments, the conjugation may comprise a non-covalent linkage of the aptamer and the solid support. In certain embodiments, the non-covalent linkage comprises an electrostatic or ionic linkage (i.e., ionic interaction between positively charged and negatively charged moieties). In certain embodiments, the non-covalent linkage comprises hydrogen bonding and/or Van der Waals (i.e., hydrophobic) interactions. A non-limiting example of non-covalent conjugation includes biotin-streptavidin non-covalent binding, wherein the aptamer is chemically modified so as to comprise a biotin moiety and the solid support is chemically modified so as to comprise a streptavidin moiety, or vice versa (i.e., aptamer-biotin and solid support-streptavidin; or aptamer-streptavidin and solid support-biotin).

In certain embodiments, the aptamer and solid support may be conjugated (i.e., covalently or non-covalently bonded) via a cross-linker. In certain embodiments, the cross-linker comprises a chemically inert alkyl or heteroalkyl moiety which is covalently bonded to the aptamer at one terminus and covalently linked to the solid support at the opposite terminus. In certain embodiments, the linker comprises a polyethylenimine (PEI) cross-linker (i.e., −[CH₂CH₂NH]m-, wherein m is an integer ranging from 1 to 10, 10 to 20, 1 to 30, 1 to 40, or 1 to 50 or more). In certain embodiments, the PEI cross-linker is linear (i.e., consists of secondary amines in the linker). In certain embodiments, the PEI cross-linker is branched (i.e., comprises one or more tertiary or quaternary amines in the linker). In certain embodiments, the linker comprises a polyethylene glycol (PEG) cross-linker (i.e., —[OCH₂CH₂]n-, wherein n is an integer ranging from 1 to 10, 1 to 20, 1 to 30, 1 to 40, or 1 to 50 or more).

### Apparatus

In another aspect, the present disclosure provides a histamine scavenging apparatus comprising a porous container having a histamine-scavenging agent held therein, wherein the porous container comprises a plurality of pores having a diameter of sufficient size such that:
(a) passage of the histamine scavenging agent therethrough is prevented; and
(b) passage of a liquid medium therethrough is permitted.

In certain embodiments, the histamine scavenging agent comprises the composition of the present disclosure (i.e., the composition comprising the histamine binding domain conjugated to a solid support). In certain embodiments, the porous container comprises nylon.

In certain embodiments, the pore diameter is selected from the group consisting of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and about 500 μm. In certain embodiments, the pore diameter is selected from the group consisting of about 5, 10, 15, 20, 25, 30, 35, 40, 45 and about 50 μm. In certain embodiments, the pore diameter is about 25 μm.

In certain embodiments, the porous container comprises a container commonly used for tea (e.g., a tea bag). In certain embodiments, the tea bag is sealed mechanically, for example using a fastener or staple, and the like. In certain embodiments, the tea bag is sealed with a sealant and/or adhesive. In certain embodiments, the sealant and/or adhesive is food-grade. In certain embodiments, the sealant and/or adhesive comprises at least one selected from the group consisting of silicone, polyurethane, modified polyacrylamide, and sealing wax. In certain embodiments, the sealant is sealing wax.

In certain embodiments, the histamine scavenging agent comprises the composition of the present disclosure.

In another aspect, the present disclosure provides a histamine scavenging apparatus comprising a column having a histamine scavenging agent held therein, an inlet for a liquid medium comprising histamine, and an outlet for a treated liquid medium with reduced histamine content, wherein the outlet comprises a porous and/or permeable material of sufficient porosity and/or permeability such that:
(a) passage of the histamine scavenging agent and optionally one or more particles within the liquid medium therethrough is prevented; and
(b) passage of the treated liquid medium therethrough is permitted.

In certain embodiments, the histamine scavenging agent comprises the composition of the present disclosure (i.e., the composition comprising the histamine binding domain conjugated to a solid support).

In certain embodiments, the porosity and/or permeability is such that the apparatus may be used as a filtration system. In certain embodiments, the apparatus may be used in combination with a filtration system. In certain embodiments, the filtration system is mobile.

### Fermented Liquids

In one aspect, the present disclosure provides methods of partially or wholly removing histamine from a fermented liquid. The terms “fermented liquid,” “liquid medium,” “fermented medium,” and “liquid media” may be used interchangeably herein. In certain embodiments, the fermented liquid is a beverage. In certain embodiments, the fermented liquid is an alcoholic beverage.

In certain embodiments, the liquid medium is selected from the group consisting of wine, beer, cider, mead, sake, vinegar, soy sauce, tepache, kombucha, and other fermented liquid beverages for human consumption. In certain embodiments, the wine is selected from the group consisting of red wine, white wine, and sparkling wine and/or champagne. In certain embodiments, the wine is red wine.

In certain embodiments, the liquid medium is contacted with at least one composition and/or apparatus of the present disclosure. In certain embodiments, the contacting provides a fermented liquid with reduced histamine content. In certain embodiments, the fermented liquid with reduced histamine content has a histamine concentration selected from the group consisting of about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.50, 2.00, 2.50, 3.00, 3.50, 4.00, 4.50, 5.00, 5.50, 6.00, 6.50, 7.00, 7.50, 8.00, 8.50, 9.00, 9.50, 10.00, 10.50, 11.00, 11.50, 12.00, 12.50, 13.00, 13.50, 14.00, 14.50, 15.00, 15.50, 16.00, 16.50, 17.00, 17.50, 18.00, 18.50, 19.00, 19.50, 20.00, 20.50, 21.00, 21.50, 22.00, 22.50, 23.00, 23.50, 24.00, 24.50, and about 25.00 mg/L.

In certain embodiments, the fermented liquid with reduced histamine content has a histamine concentration which is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% lower than that of the liquid medium before contact with the at least one composition and/or apparatus of the present disclosure. In certain embodiments, the fermented liquid with reduced histamine content has a histamine concentration which is at least 10% lower than that of the liquid medium before contact with the at least one composition and/or apparatus of the present disclosure. In certain embodiments, the fermented liquid with reduced histamine content is substantially free of histamine.

### Methods

In another aspect, the present disclosure provides a method of removing histamine from a fermented liquid, comprising contacting the fermented liquid comprising histamine with the composition of the present disclosure (i.e., the composition comprising the histamine binding domain conjugated to a solid support). The resulting product may contain histamine, but at levels substantially reduced from levels of histamine prior to treatment, or it may contain no histamine.

In another aspect, the present disclosure provides a method of treating a fermented liquid comprising histamine, the method comprising contacting the fermented liquid with a histamine scavenging agent to provide a fermented liquid with reduced histamine content and a histamine adsorbed species.

In certain embodiments, the method of treating a fermented liquid comprising histamine at least partially removes histamine from the fermented liquid. In certain embodiments, the only component of the fermented liquid which is at least partially removed is histamine. In certain embodiments, sulfites present in the fermented liquid are not substantially removed and/or degraded.

In certain embodiments, the histamine scavenging agent comprises the composition of the present disclosure.

In certain embodiments, the contacting occurs by immersion of the histamine scavenging agent in the fermented liquid. In certain embodiments, the immersion occurs for a period of time selected from the group consisting of about 1, 2, 3, 4, 5, 10, 15, 20, 25, 30, 35, 40, 45, 50, 55, and about 60 min. In certain embodiments, the period of time of immersion may exceed 1 hour.

In certain embodiments, the contacting occurs with agitation. In certain embodiments, the agitation comprises shaking and/or stirring.

In certain embodiments, the contacting occurs by passage of the fermented liquid through a stationary phase comprising the histamine scavenging agent.

In certain embodiments, the fermented liquid is alcoholic or non-alcoholic. In certain embodiments, the fermented liquid is selected from the group consisting of wine, beer, cider, mead, sake, vinegar, soy sauce, tepache, kombucha, and other fermented liquid beverages for human consumption.

In certain embodiments, the fermented liquid with reduced histamine content has a histamine concentration selected from the group consisting of about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.50, 2.00, 2.50, 3.00, 3.50, 4.00, 4.50, 5.00, 5.50, 6.00, 6.50, 7.00, 7.50, 8.00, 8.50, 9.00, 9.50, 10.00, 10.50, 11.00, 11.50, 12.00, 12.50, 13.00, 13.50, 14.00, 14.50, 15.00, 15.50, 16.00, 16.50, 17.00, 17.50, 18.00, 18.50, 19.00, 19.50, 20.00, 20.50, 21.00, 21.50, 22.00, 22.50, 23.00, 23.50, 24.00, 24.50, and about 25.00 mg/L.

In certain embodiments, the fermented liquid with reduced histamine content has a histamine concentration which is at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% lower than that of the liquid medium before contact with the at least one composition and/or apparatus of the present disclosure. In certain embodiments, the fermented liquid with reduced histamine content has a histamine concentration which is at least 10% lower than that of the liquid medium before contact with the at least one composition and/or apparatus of the present disclosure. In certain embodiments, the fermented liquid with reduced histamine content is substantially free of histamine.

In certain embodiments, consumption of the fermented liquid with reduced histamine content does not cause any symptoms commonly associated with histamine intolerance in a subject. In certain embodiments, the fermented liquid with reduced histamine content has a taste which is essentially identical to the taste of the fermented liquid comprising histamine.

In certain embodiments, the method further comprises separating the fermented liquid with reduced histamine content and the histamine adsorbed species. In certain embodiments, the histamine adsorbed species comprises a magnetic solid support. In certain embodiments, the solid support is a bead, wherein the bead optionally comprises steel. In certain embodiments, the histamine adsorbed species is separated from the fermented liquid with reduced histamine content by magnetic separation.

In certain embodiments, the method further comprises regenerating the histamine scavenging agent, comprising the steps of:
(a) contacting the histamine adsorbed species with a low pH solution to provide a first washed species; and
(b) contacting the first washed species with a buffered solution to provide the histamine scavenging agent.

In certain embodiments, the low pH solution comprises a glycine solution. In certain embodiments, the glycine solution comprises a glycine solution having a glycine concentration of about 0.05 M to about 0.5 M. In certain embodiments, the low pH solution has a pH of about 2.2. In certain embodiments, the buffered solution comprises a phosphate-buffered saline (PBS) solution. In certain embodiments, the buffer solution comprises an ethylenediaminetetraacetic acid (EDTA) solution.

In certain embodiments, the method of regenerating the histamine scavenging agent comprising, in part, contacting the histamine adsorbed species with the low pH solution is suitable for histamine scavenging compositions comprising antibody-conjugated histamine scavenging agents.

In non-limiting alternative embodiments, wherein the histamine scavenging composition comprises an aptamer-conjugated histamine scavenging agent, alternative methods of regenerating the histamine scavenging agent may be utilized (e.g., utilizing a denaturant solution).

In such embodiments, the method further comprises regenerating the histamine scavenging agent, comprising the steps of:
(a) contacting the histamine adsorbed species with a denaturant to provide a first washed species; and
(b) contacting the first washed species with a buffered solution to provide the histamine scavenging agent.

In certain embodiments, the denaturant comprises a urea solution. In certain embodiments, the urea solution has a urea concentration ranging from about 1 M to about 8 M. In certain embodiments, the buffered solution comprises a phosphate-buffered saline (PBS) solution. In certain embodiments, the buffer solution comprises an ethylenediaminetetraacetic acid (EDTA) solution.

In certain embodiments, the histamine scavenging agent is regenerated without a loss in histamine scavenging efficiency. In certain embodiments, the histamine scavenging agent is stored in a suspension buffer solution after regeneration. In certain embodiments, the suspension buffer solution comprises a 20% ethanol (EtOH) phosphate-buffered saline (PBS) solution. In certain embodiments, the histamine scavenging agent can be stored long term in a 20% EtOH phosphate-buffered saline (PBS) solution. In certain embodiments, the histamine scavenging agent can be stored long term in an ethylenediaminetetraacetic acid (EDTA) solution.

In certain embodiments, the histamine scavenging agent is washed with the buffered solution prior to a subsequent use in histamine removal from a second fermented liquid.

In another aspect, the present disclosure provides a method of at least partially removing histamine from a fermented liquid using the kit of the present disclosure to provide a fermented liquid with reduced histamine content, the method comprising:
contacting a parcel comprising the histamine scavenging composition (i.e., the composition comprising the histamine binding domain conjugated to a solid support) with the fermented liquid to provide a fermented liquid with reduced histamine content and a parcel comprising a histamine adsorbed species;
separating the fermented liquid with reduced histamine content and the parcel comprising the histamine adsorbed species;
contacting the parcel comprising the histamine adsorbed species with the low pH or denaturant solution to provide a parcel comprising a first washed species;
contacting the parcel comprising the first washed species with the wash buffer solution to regenerate the parcel comprising the histamine scavenging composition; and
optionally storing the parcel comprising the histamine scavenging composition in the stored suspension buffer.

In certain embodiments, the regenerated parcel comprising the histamine scavenging composition is used to at least partially remove histamine from a fermented liquid. In certain embodiments, the parcel comprising the histamine scavenging composition is separated from the suspension buffer and contacted with the wash buffer solution.

In certain embodiments, the fermented liquid is a fermented liquid beverage for human consumption. In certain embodiments, the beverage for human consumption is selected from the group consisting of wine, beer, cider, mead, sake, vinegar, soy sauce, tepache, and kombucha. In certain embodiments, the wine is red wine.

### Kits

In another aspect, the present disclosure provides a kit comprising the composition and/or apparatus of the present disclosure, and instructional material for use thereof, wherein the instructional material comprises instructions for at least partially removing histamine from a fermented liquid.

In one aspect, the kit comprises a parcel containing the histamine scavenging composition of the invention, a low pH solution, a wash buffer solution, and a suspension buffer solution. In one embodiment, the low pH solution comprises a glycine solution, which certain embodiments, has a glycine concentration of about 0.05 M to about 0.5 M. In other embodiments, the low pH solution has a pH of about 2.2. In other embodiments, the wash buffer solution comprises a phosphate-buffered saline (PBS) solution. In yet other embodiments, the suspension buffer solution comprises a 20% EtOH phosphate-buffered saline (PBS) solution. In yet other embodiments, the suspension buffer comprises an ethylenediaminetetraacetic acid (EDTA) solution. In certain embodiments, the kit comprising the low pH solution and a histamine scavenging composition comprises an antibody-conjugated histamine scavenging agent.

In one aspect, the kit comprises a parcel containing the histamine scavenging composition of the invention, a denaturant solution, a wash buffer solution, and a suspension buffer solution. In one embodiment, the urea solution has a urea concentration ranging from about 1 M to about 8 M. In other embodiments, the wash buffer solution comprises a phosphate-buffered saline (PBS) solution. In yet other embodiments, the suspension buffer solution comprises a 20% EtOH phosphate-buffered saline (PBS) solution. In certain embodiments, the kit comprising the urea solution and a histamine scavenging composition comprises an aptamer-conjugated histamine scavenging agent.

In another aspect, the invention includes a method of at least partially removing histamine from a fermented liquid using the kit as disclosed herein, to provide a fermented liquid with reduced histamine content. The method comprises contacting a parcel comprising the histamine scavenging composition with the fermented liquid to provide a fermented liquid with reduced histamine content and a parcel comprising a histamine adsorbed species; separating the fermented liquid with reduced histamine content and the parcel comprising the histamine adsorbed species; contacting the parcel comprising the histamine adsorbed species with the low pH solution to provide a parcel comprising a first washed species; contacting the parcel comprising the first washed species with the wash buffer solution to regenerate the parcel comprising the histamine scavenging composition; and optionally storing the parcel comprising the histamine scavenging composition in the stored suspension buffer.

In one embodiment, the regenerated parcel comprising the histamine scavenging composition is used to at least partially remove histamine from a second fermented liquid. In another embodiment, the parcel comprising the histamine scavenging composition is separated from the suspension buffer and contacted with the wash buffer solution. In yet another embodiment, the fermented liquid is a fermented liquid beverage for human consumption, which in another embodiment, is selected from the group consisting of wine, beer, cider, mead, sake, vinegar, soy sauce, tepache, and kombucha. In yet an additional embodiment, the wine is red wine.

### EXAMPLES

Various embodiments of the present application can be better understood by reference to the following Examples which are offered by way of illustration. The scope of the present application is not limited to the Examples given herein.

### Example 1: Preparation of a Histamine Scavenging Apparatus

### Antibody Histamine Scavenging Agent

A cross-linking buffer is prepared (50 mM Tris-HCl, 150 mM NaCl, 5 mM EDTA, 0.1% NP-40). Protein A sepharose beads are washed with distilled water. Beads are centrifuged at 500 g for 2-5 mins at room temperature with subsequent removal of supernatant. This process is repeated approximately three times, then the beads are suspended in the cross-linking buffer.

The antibody is diluted to a concentration of 0.5 mg/mL in cross-linking buffer, then the antibody is added to the suspension comprising the protein A sepharose beads, and incubated overnight. The beads are washed with cross-linking buffer and centrifuged at 500 g for 2-5 mins at room temperature. This process is repeated approximately three times. Next, the beads are washed once more using Dulbecco's PBS and centrifuged at 500 g for 2-5 min at room temperature.

Disuccinimidyl suberate (DSS, 10 mg) was dissolved in DMSO (0.5 mL) and immediately diluted 10-fold in Dulbecco's PBS (4.5 mL). Next, the beads were resuspended in the DSS/PBS solution (5 mL). The beads were incubated for about 1 hour at room temperature with occasional shaking and/or agitation.

The beads were collected by centrifugation at 500 g for 2-5 min and the DSS/PBS solution was aspirated. The beads were resuspended in 100 mM glycine-HCl (pH 2.7) and the beads were collected by centrifugation at 500 g for 2-4 min, with removal of the supernatant. The beads were washed twice with cross-linking buffer, and subsequently resuspended in PBS with 20% ethanol for long term storage.

### Example 2: Removal of Histamine by Steeping with Composition and/or Apparatus of the Present Disclosure

In certain embodiments, the histamine scavenging composition of the present disclosure is held in a porous container, comprising an apparatus of the present disclosure. In certain embodiments, the histamine scavenging composition can have any one of a number of shapes, including spherical (e.g., a bead), or may comprise a strip, plate, disk, rod, non-spherical particle, tube, and the like. The average particle size of the composition of the present disclosure may vary. However, the pore size of the porous container is correlated with the average particle size such that, upon immersion and/or contact with a liquid medium, passage of the composition therethrough is prevented and passage of the liquid medium is permitted. In certain embodiments, the porous container is analogous to a tea bag.

In certain embodiments, the porous container comprises a suitable material (e.g., nylon woven mesh and/or polypropylene non-woven mesh). In non-limiting embodiments, the porous container has a pore size of less than about 10 μm and the histamine scavenging composition has a diameter of greater than about 10 μm. In further non-limiting embodiments, the porous container has a pore size of less than about 25 μm and the histamine scavenging composition has a diameter of greater than about 25 μm. In further non-limiting embodiments, the porous container has a pore size of less than about 50 μm and the histamine scavenging composition has a diameter of greater than about 50 μm. In further non-limiting embodiments, the porous container has a pore size of less than about 100 μm and the histamine scavenging composition has a diameter of greater than about 100 μm. In further non-limiting embodiments, the porous container has a pore size of less than about 150 μm and the histamine scavenging composition has a diameter of greater than about 150 μm. In further non-limiting embodiments, the porous container has a pore size of less than about 200 μm and the histamine scavenging composition has a diameter of greater than about 200 μm. In further non-limiting embodiments, the porous container has a pore size of less than about 250 μm and the histamine scavenging composition has a diameter of greater than about 250 μm. In further non-limiting embodiments, the porous container has a pore size of less than about 300 μm and the histamine scavenging composition has a diameter of greater than about 300 μm. In further non-limiting embodiments, the porous container has a pore size of less than about 350 μm and the histamine scavenging composition has a diameter of greater than about 350 μm. In further non-limiting embodiments, the porous container has a pore size of less than about 400 μm and the histamine scavenging composition has a diameter of greater than about 400 μm.

The porous container-bound composition may be used to remove histamine from a fermented liquid, or any liquid comprising histamine. In certain embodiments, the anti-histamine antibody selectively binds to histamine in an aqueous solution. In certain embodiments, the aqueous solution may further comprise alcohol (i.e., fermented alcoholic beverage).

The composition may be immersed (i.e., steeped) in the histamine containing solution for a period of time suitable to permit binding of histamine to the histamine binding domain (e.g., histamine binding aptamer and/or histamine binding antibody). The composition may be immersed in a beverage prior to consumption, non-limiting examples including a glass and/or bottle of wine, beer, sake, mead, champagne, kombucha, and/or tepache. In certain embodiments, the immersion may occur during a fermentation process. In certain embodiments, the immersion may occur after a fermentation process.

In certain embodiments, the binding of histamine to the histamine binding domain is instantaneous. In certain embodiments, the binding may require an amount of time of about 1 min to about 60 mins. The amount of time required for binding is dependent upon the concentration of histamine in the liquid medium, the amount and/or concentration of histamine binding domain conjugated to the solid support, the volume of the liquid medium, and the surface area of the solid support.

In certain embodiments, the liquid medium and scavenging agent are agitated as to facilitate diffusion within the liquid medium. In certain embodiments, the agitation comprises stirring and/or shaking.

Once a sufficient period of time has been allotted for binding of histamine to the histamine scavenging agent (e.g., a histamine binding aptamer conjugated to a solid support or an anti-histamine antibody conjugated to a solid support) the used scavenging agent and treated liquid medium may be separated.

In certain embodiments, the used scavenging agent (i.e., histamine adsorbed species) comprises a plurality of histamine binding aptamers conjugated to a solid support, wherein the histamine binding aptamers are at least partially saturated with bound histamine. In certain embodiments, the used scavenging agent comprises a plurality of anti-histamine antibodies conjugated to a solid support, wherein the anti-histamine antibodies are at least partially saturated with bound histamine. The bound histamine may be removed from the composition, as described elsewhere herein, to regenerate the scavenging ability of the composition. The regenerated histamine scavenging agent may be used repeatedly without a decrease in efficiency with each iterative regeneration.

In certain embodiments, the treated liquid medium has a reduced histamine content. In certain embodiments, the treated liquid medium is substantially free of histamine. In certain embodiments, the treated liquid medium is consumed directly. In other embodiments, the treated liquid medium is packaged without substantial decomposition.

### Example 3: Removal of Histamine by Filtration of Fermented Liquid with Composition of the Present Disclosure

In certain embodiments, the histamine scavenging composition of the present disclosure comprises a stationary phase. In certain embodiments, the stationary phase can have any one of a number of shapes, including spherical (e.g., a bead), or may comprise a strip, plate, disk, rod, non-spherical particle, tube, well, and the like. The composition of the present disclosure may be packed in a column and/or funnel to form a porous matrix. The porous matrix may be supported at the outlet valve by an additional porous material (e.g., ground glass frit or filter). A fermented liquid medium comprising histamine may be passed through the column and/or funnel, thereby contacting the composition of the present disclosure, and substantially reducing and/or removing the histamine from the liquid medium.

The flow rate of the liquid medium through the column and/or funnel may be adjusted in a number of ways, including manipulation of particle size, outlet filter and/or frit porosity, outlet diameter, and pressure (positive or negative), among others. The average diameter of the composition of the present disclosure may vary. Similarly, the degree of packing of the matrix (i.e., matrix density) and may be varied as desired to control flow rate. However, the particle size of the composition is correlated with the pore size of the supporting porous material at the outlet valve, such that, upon passage of the liquid medium therethrough, passage of the composition therethrough is prevented and passage of the liquid medium therethrough is permitted (i.e., pore size of the outlet filter/frit is less than the particle size of the stationary phase).

In certain embodiments, one or more particles (e.g., sediment and/or yeast) in the liquid medium may be filtered prior to, during, and/or after contact with the composition of the present disclosure. In certain embodiments, the pore size of the supporting porous material and/or the particle size of the composition are such that one or more particles in the liquid medium may be filtered (i.e., separated from the liquid with reduced histamine content) concurrently with histamine scavenging.

In certain embodiments, the flow rate is correlated with the amount of time necessary to sufficiently bind and/or remove histamine from the liquid medium, such that upon elution of the treated liquid medium from the outlet of the column and/or funnel, the histamine is absent or substantially reduced. Similarly, the properties of the composition of the present disclosure (e.g., particle size, histamine binding aptamer and/or anti-histamine antibody loading, and surface area, etc.) may be manipulated to alter the amount of time necessary for the contacting of the liquid medium to result in removal of histamine prior to elution.

However, after a number of uses, the composition of the present disclosure will become saturated with histamine (i.e., solid support conjugated histamine binding aptamer and/or anti-histamine antibodies become at least partially saturated with histamine). The number of uses necessary to achieve partial or total saturation will vary depending on the aforementioned properties of the composition, in addition to the volume of liquid medium and concentration of histamine in the liquid medium which has contacted the composition.

Once saturated with histamine, the histamine scavenging composition may be regenerated in manner analogous to that which has described elsewhere herein. Thus, the histamine may be removed from the composition, as described elsewhere herein, to regenerate the scavenging ability of the composition.

In certain embodiments, the treated liquid medium has reduced histamine content. In certain embodiments, the treated liquid medium is substantially free of histamine. In certain embodiments, the treated liquid medium is consumed directly. In other embodiments, the treated liquid medium is packaged without substantial decomposition.

The column and/or funnel described herein is suitable for use on either large or small scale. For example, the filter may be used at a business establishment or private residence in which individual beverages are served (e.g., a glass or bottle of wine). Alternatively, a large scale column may be used at an industrial facility during the processing of large volumes of fermented liquids.

### Example 4: Regeneration of the Histamine Scavenging Activity of the Composition of the Present Disclosure

Regardless of the properties (e.g., surface area or amount of histamine binding domain conjugated to solid support) or particular apparatus (e.g., bag, funnel, or column), after a sufficient number of iterative uses, the histamine-binding and/or scavenging efficiency of the material will decrease, as fewer anti-histamine antibodies on the surface of the composition are available for binding of histamine in the liquid medium with each use.

However, as mentioned elsewhere herein, the histamine bound to the histamine binding domains may be removed, thereby restoring the histamine scavenging activity of the composition. Furthermore, this regeneration is applicable to the composition described herein independent of the apparatus in which the composition is utilized.

For example, if the composition is used in a porous container (e.g., tea bag), after a sufficient number of uses, the material may be regenerated as described herein. First, the tea bag comprising the histamine scavenging composition is washed with a low pH solution for a short period of time, a non-limiting example including washing with a 0.1 M glycine (pH 2.2) solution for about 5 s. Alternatively, for embodiments of the invention in which the histamine scavenging composition comprises an aptamer, the tea bag comprising the histamine scavenging composition is washed with a denaturant solution for a short period of time, a non-limiting example including washing with a 8.0 M urea solution for about 5 s. Next, the composition is further washed with buffered solution. Non-limiting examples include washing with a phosphate-buffered saline (PBS) solution for about 10 s. Finally, the composition may be stored for an iterative use in a storage solution. Non-limiting examples of a storage solution include a solution comprising 20% EtOH in PBS. Non-limiting examples of storage conditions include reduced temperature. In certain embodiments, the reduced temperature is about 3° C. to about 12° C. In certain embodiments, prior to subsequent use after storage, the composition may be washed one or more times with water and optionally drained.

Alternatively, if the composition is used as a stationary phase in a column or funnel, after a sufficient number of uses, the material may also be regenerated as described herein. First, a sufficient number of column volumes of a solution of a low pH solution, non-limiting examples including 0.1 M glycine (pH 2.2), is passed through the column. Alternatively, a sufficient number of column volumes of a denaturant solution, non-limiting examples including a 8.0 M urea solution, is passed through the column. Next, a sufficient number of column volumes of a buffered solution, non-limiting examples including a PBS solution, is passed through the column. Finally, the composition may be stored as a wet-packed column. Non-limiting examples of a solution for wet-packed storage include a solution of 20% EtOH in PBS, preferably at a reduced temperature (about 3° C. to about 12° C.). In certain embodiments, wherein the histamine binding domain comprises an aptamer, the composition may be stored in an ethylenediaminetetraacetic acid (EDTA) solution. In certain embodiments, prior to subsequent use after storage, the composition may be washed one or more times with water and optionally drained.

### Example 5: Quantification of the Histamine Scavenging Activity of an Exemplary Embodiment of the Composition of the Present Disclosure

The present disclosure provides exemplary data indicating the utility of a non-limiting embodiment of the present invention (i.e., histamine scavenging composition described in Example 1) in the at least partial removal of histamine from a beverage, even after an extended period of repeated use (i.e., >1 year of repeated use).

The general procedure for the experiments described herein comprises contacting the composition with the histamine containing beverage for a period of about 15 minutes, then washing the composition as described elsewhere herein.

The exemplary data provided herein demonstrate the ability of the composition to remove histamine from red wine consistently in each treatment. Mondavi Cabernet Sauvignon wine was treated 3 times and the amount of histamine removed during each treatment was quantified by LC/MS/MS by analysis of both the wine and the washing solution after contact with the composition. Positive controls (e.g., white grape juice (WGJ) with added histamine) were used to confirm the reliability of the histamine quantification. The results disclosed herein indicate that approximately 81 μm of histamine in 100 mL of wine is removed with each period of contact (Table 3).

TABLE 3
Histamine content of solvents during the process of histamine removal

| | | | | Histamine |
|---|---|---|---|---|
| | | | | concentration |
| Trial | Treatment | Solvent | Description | (ng/mL) |
| 1 | 0 | Red wine | Untreated wine (Mondavi | 6790 |
| | | | Cabernet Sauvignon) | |
| 1 | 2 | Glycine | Histamine eluted during wash | 760 |
| | | | step after 2^{nd} treatment | |
| 1 | 3 | Glycine | Histamine eluted during wash | 635 |
| | | | step after 3^{rd} treatment | |
| 1 | 3 | Red wine | Wine after 3^{rd} treatment | 4290 |
| 2 | 1 | Red wine | Wine after 1^{st} treatment | 5410 |
| 2 | 1 | Glycine | Histamine eluted during wash | 894 |
| | | | step after 1^{st} treatment | |
| 2 | 2 | Red wine | Wine after 2^{nd} treatment | 4620 |
| 2 | 2 | Glycine | Histamine eluted during wash | 896 |
| | | | step after 2^{nd} treatment | |
| 2 | 3 | Red wine | Wine after 3^{rd} treatment | 4020 |
| 2 | 3 | Glycine | Histamine eluted during wash | 704 |
| | | | step after 3^{rd} treatment | |
| 3 | N/A | WGJ | Positive control, 60 μg/mL histamine | 48230^{a} |
| 3 | N/A | WGJ | Positive control, 6 μg/mL histamine | 4690 |
| 3 | N/A | WGJ | Positive control, 6 μg/mL histamine | 4813 |
| 3 | N/A | WGJ | Positive control, 600 ng/mL histamine | 462 |
| 3 | N/A | WGJ | Positive control, 60 ng/mL histamine | 110 |
| 3 | N/A | WGJ | Negative control (no histamine) | 42^{b} |

^{a}Above limit of detection;
^{b}Below limit of detection

Measurement of several samples from the experiment described herein were repeated to confirm consistency of histamine measurement (Table 4).

TABLE 4
Histamine content of solvents during the process
of histamine removal (duplicate data)

| | | | | Histamine |
|---|---|---|---|---|
| | | | | concentration |
| Trial | Treatment | Solvent | Description | (ng/mL) |
| 1 | 0 | Red wine | Mondavi Cabernet Sauvignon, untreated | 6799 |
| 1 | 1 | Red wine | Wine after 1^{st} treatment | 5726 |
| 1 | 1 | Glycine | Histamine eluted from wash | 897 |
| | | | step after 1^{st} treatment | |
| 1 | 2 | Red wine | Wine after 2^{nd} treatment | 4750 |
| 1 | 2 | Glycine | Histamine eluted from wash | 900 |
| | | | step after 2^{nd} treatment | |
| 1 | 3 | Red wine | Wine after 3^{rd} treatment | 4081 |
| 2 | 3 | Red wine | Wine after three treatments | 4404 |
| 3 | N/A | WGJ | Positive control, 60 ng/mL histamine | 71 |
| 3 | N/A | WGJ | Positive control, 60 ng/mL histamine | 70 |
| 3 | N/A | WGJ | Positive control, 242 ng/mL histamine | 233 |
| 3 | N/A | WGJ | Positive control, 242 ng/mL histamine | 248 |
| 3 | N/A | WGJ | Positive control, 2416 ng/mL histamine | 2035 |
| 3 | N/A | WGJ | Positive control, 2416 ng/mL histamine | 1960 |
| 4 | N/A | Red wine | Sangiovese, untreated | 1159 |
| 4 | N/A | Red wine | Nebbiolo, untreated | 283 |

The present disclosure further provides exemplary data regarding histamine content of several types of wine before and after multiple treatments with the exemplary embodiment of the present disclosure (Table 5).

TABLE 5
Histamine content of wine before and after treatment

| | Histamine | | Histamine | | |
|---|---|---|---|---|---|
| | Content | | Content | | No. of |
| Wine | (untreated) | | (treated) | | Treatments |
| Mondavi Cabernet | 6270 | ng/mL | 1860 | ng/mL | 10 |
| Sauvignon | | | | | |
| Sangiovese, Chianti | 1070 | ng/mL | 730 | ng/mL | 3 |
| Classico Volpaia | | | | | |
| Nebbiolo, G.D. | 390 | ng/mL | 280 | ng/mL | 3 |
| Vajra, Langhe | | | | | |
| Scribe Pinot Noir, CA | 400 | ng/mL | 250 | ng/mL | 3 |

### General Procedure for LC MS

The experiments described herein were performed according to the commercial instructions for LC/MS provided by Agilent Technologies (Quick, Easy and Reliable Detection of Histamine in Food Using the Agilent 6490 Triple Quadrupole LC/MS with Jet Stream Technology) with minor modifications to optimize analysis response and retention time.

### Reagents and Standards

Formic acid in water (0.1%) was prepared by diluting 1 mL of formic acid to 1000 mL in water. This reagent was used for both the HPLC mobile phase (MP-A) and sample diluent.

Formic acid in acetonitrile (0.1%) was prepared by diluting 1 mL of formic acid to 1000 mL in acetonitrile. This reagent is used for both the HPLC and mobile phase (MP-B).

A solution comprising formic acid in acetonitrile and water (60% water: 40% ACN; 0.1% formic acid) was prepared by adding 60 mL of 0.1% formic acid in water with 40 mL of 0.1% formic acid in acetonitrile. This solution was used as a diluent for all samples.

A solution of HCl in water (0.1%) was prepared by adding 0.85 mL of concentration HCl to about 70 mL of water and adjusting the volume to 100 mL. This solution was used for solubilizing the standard and internal standard material.

### Reference Material Preparation

For quantitation, a calibration curve was prepared using known quantities of a certified reference standard of Histamine dihydrochloride (Supelco Inc., Bellefonte, PA 16823). All measured weights are converted to the histamine free base. Histamine-d₄ dihydrochloride (Sigma-Aldrich, St. Louis. MO 68178) is similarly prepared as the internal standard solution (1 ng/μL).

Reference solutions were prepared as follows:
(a) Primary calibration solution was prepared for use in the calibration curve. This was prepared at a concentration of 1 mg/mL in 0.1% HCl in water. Subsequent dilutions were performed using this solution.
(b) Secondary source of histamine reference material was purchased for preparation of bi-level quality controls. This was also prepared at a concentration of 1 mg/mL in 0.1% HCL in water.
(c) Powder is weighed out an analytical balance and added into solvent (i.e., 0.1% HCl in water) at a concentration of 1 mg/mL. This is the stock standard solution.

Substock solutions are prepared as follows: Substock A—10 ng/μL (a 1+99 dilution of the stock solution); Substock B—1 ng/μL (a 1+9 dilution of Substock A); Substock C—0.1 ng/μL (a 1+9 dilution of Substock B); The difference in molecular weight for the salt and free-base is taken into account in solution preparation.

### Histamine Internal Standard Preparation (Histamine-d₄)

Deuterated histamine dihydrochloride (histamine-d₄) was purchased for preparation of the internal standard solution. This was prepared at a concentration 1 ng/μL in aq. HCl (0.1%).

### Sample Preparation

After equilibration of the wine to room temperature, wine containers are mixed and approximately 1000 μL of the wine is sampled. This sample is filtered by passing it through a 0.2-μm nylon filter (Whatman™ part #6789-1302) using a 1 mL Luer lock sterile syringe (Fisher Scientific part #14955464) to supply pressure. Filtered wine (0.1 mL) is aliquoted and diluted with 1 mL of mobile phase. This mobile phase is a mixture of water and acetonitrile (60%: 40%) in 0.1% formic acid. An internal standard (50 μL of histamine-d₄) at a concentration of 1 ng/μL in 0.1% HCl in water) is added to all the samples (excluding the mobile phase blanks and matrix blank).

### Calibration Curve

Red grape juice was chosen as a suitable matrix blank for the analysis because of its similarity to wine with regard to consistency and pH. Red grape juice is generally very low in histamine concentrations. A calibration curve is prepared in red grape juice and mobile phase (60:40 water:acetonitrile containing 0.1% formic acid). Blank matrix (100 μL) and 60:40 mobile phase (1 mL) is added to each calibrator and blank. The default sample volume is 0.1 mL. The lower-limit of detection for the analysis was 5 ng/ml.
Calibration 1—2000 ng/mL; 200 μL substock A; 50 μL ISTD (histamine-d₄ 1 ng/μL)
Calibration 2—1000 ng/ml; 100 μL substock A; 50 μL ISTD (histamine-d₄ 1 ng/μL)
Calibration 3—500 ng/ml; 50 μL substock A; 50 μL ISTD (histamine-d₄ 1 ng/μL)
Calibration 4—200 ng/ml; 20 μL substock A; 50 μL ISTD (histamine-d₄ 1 ng/μL)
Calibration 5—50 ng/mL; 50 μL substock B; 50 μL ISTD (histamine-d₄ 1 ng/μL)
Calibration 6—20 ng/ml; 20 μL substock B; 50 μL ISTD (histamine-d₄ 1 ng/μL)
Calibration 7—5 ng/ml; 50 μL substock C; 50 μL ISTD (histamine-d₄ 1 ng/μL)
Blank—0 ng/ml; 50 μL ISTD (histamine-d₄ 1 ng/μL)

### Instrumentation

Agilent 1200 series LC system-Agilent 6430 Triple Quadrupole LC/MS system. (Agilent Technologies 5301 Stevens Creek Blvd. Santa Clara, CA 95051). Analytical column: Cogent Diamond Hydride column 100 Å 4 μm 150×2.1 mm 7000 15P-2 (Cornerstone Scientific 8209 Market Street Wilmington, NC 28411); Column temperature: 40° C.; Injection volume: 2 μL; Mobile phase A: water, 0.1% formic acid; Mobile phase B: acetonitrile, 0.1% formic acid; Flow rate: 0.4 mL/min; Post time: 1 min; Gradient: t₀=40% solvent B; t_{3 min}=10% solvent B; t_{5.5 min}=10% solvent B; t_{6.0 min}=40% solvent B; t_{9.5 min}=40% solvent B;

MS Acquisition parameters: ESI with Agilent Jet Stream, position ionization mode; Sheath gas temperature: 350° C.; Sheath gas flow rate: 12 L/min; Drying gas flow rate: 11 L/min; Drying gas temperature: 200° C.; Nebulizer pressure: 50 psi; Nozzle voltage: 1.25 kV; Vcap voltage: 5.5 kV

### Data Acquisition

The prepared samples are injected directly into the LC/MS/MS. The HPLC mode is HILIC. The mass spectrometer is operated in positive ion electrospray mode using MRM data acquisition.

TABLE 6
Acquisition parameters for histamine showing the MRM transitions

| | Precursor | Product | Collision | Cell |
|---|---|---|---|---|
| Target analytes | ion (m/z) | ion (m/z) | energy | acceleration |
| Histamine | 112 | 68.1 | 24 | 7 |
| (Quantifier) | | | | |
| Histamine | 112 | 95.1 | 16 | 7 |
| Histamine D4 | 116 | 99.1 | 12 | 7 |
| Histamine D4 | 116 | 85.1 | 16 | 7 |

### Quantitative Analysis

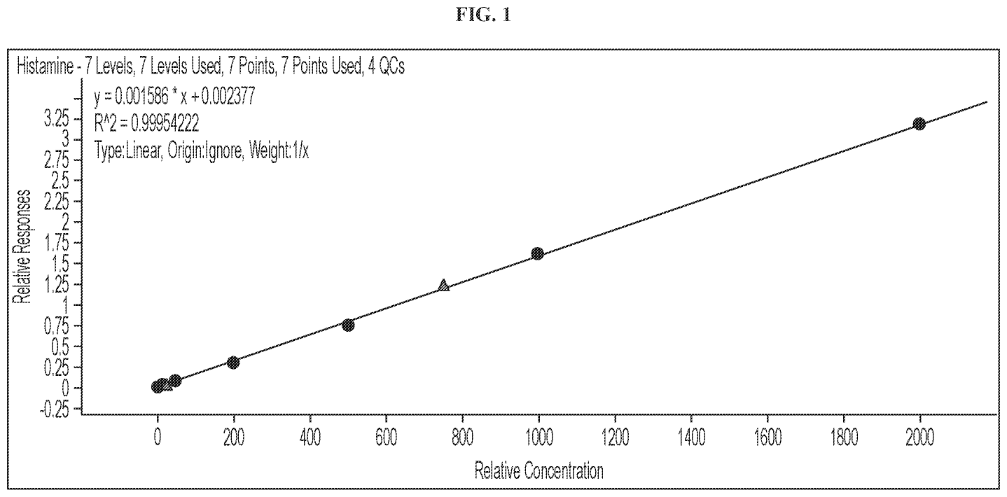

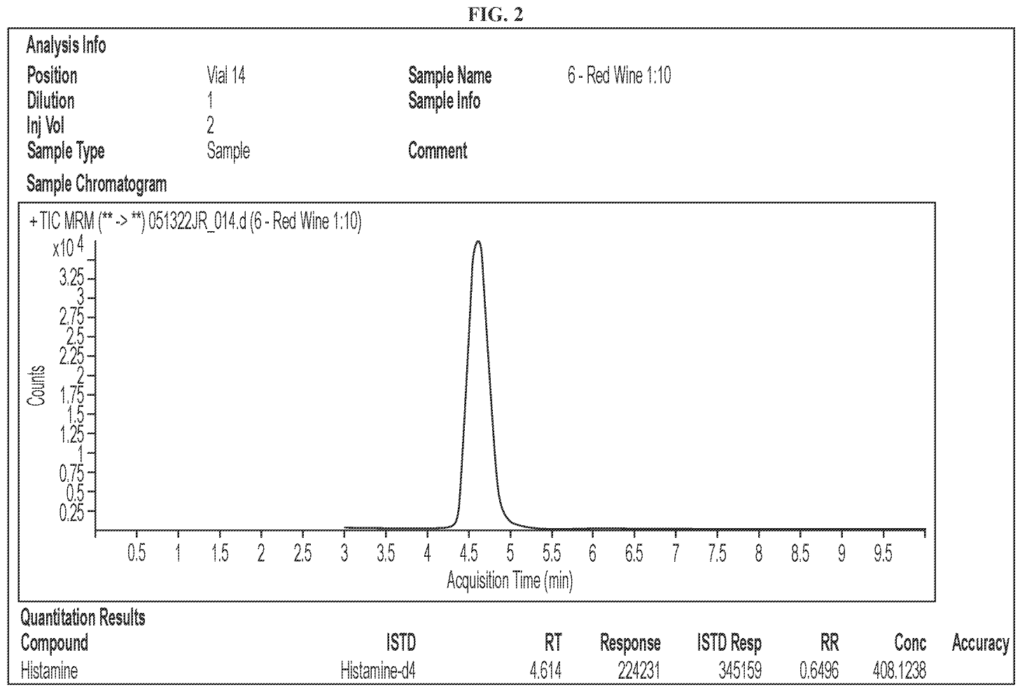

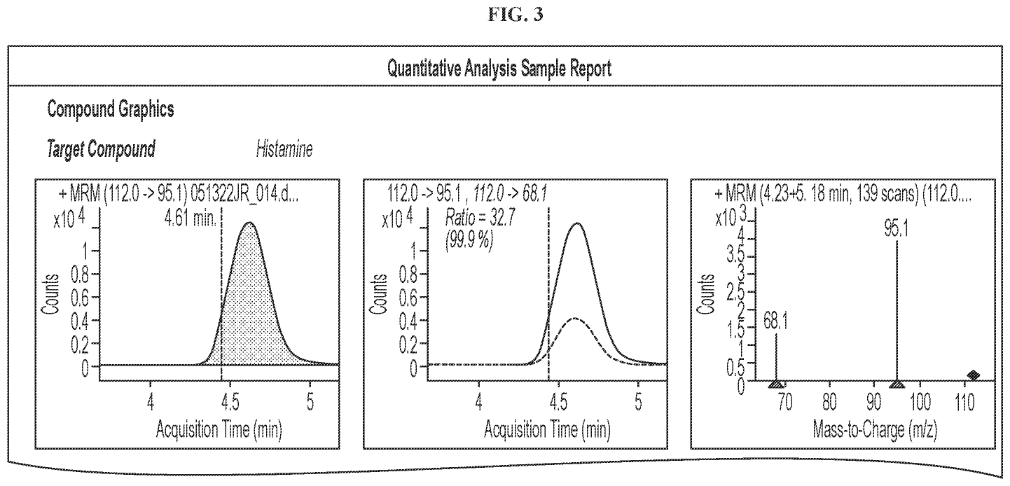

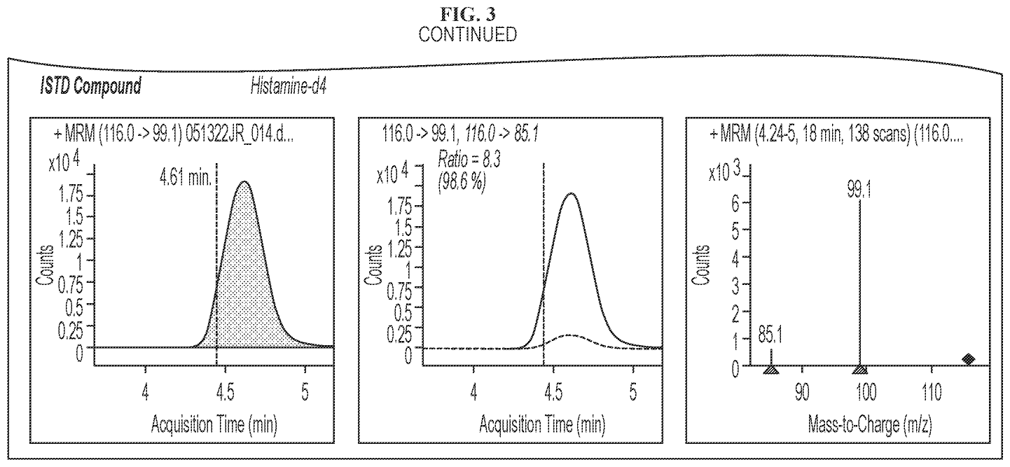

For each injection, the response ratio is calculated as described herein. The peak area of the transition (112.0>95.1) for the analyte divided by the peak are of the transition (116.0>99.1) to determine the peak area ratio. A calibration curve is constructed by plotting the peak area ratio (y-axis) against the concentration for each calibration level (x-axis). A linear weighted (1/x) calibration curve is generated from this data (FIG. 1). Representative integrated chromatograms and mass spectra are provided for histamine in wine (FIGS. 2-3).

### Quality Control

Two levels of quality control were performed for each batch of samples. These were prepared fresh with each batch from the secondary source of histamine dihydrochloride solution (1 mg/mL) (Sigma Aldrich®). Quality control sample concentrations are prepared at 25 ng/ml and 750 ng/mL. These were prepared for the red grape juice blank. These calibrators are injected at the beginning and end for each batch. Acceptable values must fall within +20% of the target value.

### Enumerated Embodiments

The following exemplary embodiments are provided, the numbering of which is not to be construed as designating levels of importance:

Embodiment 1 provides a composition comprising a histamine binding domain conjugated to a solid support.

Embodiment 2 provides the composition of Embodiment 1, wherein the histamine binding domain is selected from the group consisting of an aptamer, a histamine binding fragment (Fab), a single-chain variable fragment (scFv), a single-domain antibody (sdAb), an anti-histamine antibody, and an anticalin.

Embodiment 3 provides the composition of Embodiment 2, wherein the histamine binding domain is an aptamer.

Embodiment 4 provides the composition of Embodiment 3, wherein the aptamer comprises a DNA oligonucleotide.

Embodiment 5 provides the composition of Embodiment 4, wherein the DNA oligonucleotide shares at least 85%, 90%, 95%, 96%, 97%, 98%, or 99% sequence homology with a DNA oligonucleotide selected from the group consisting of A1 (SEQ ID NO:1), A2 (SEQ ID NO: 2), A3 (SEQ ID NO:3), A4 (SEQ ID NO:4), A5 (SEQ ID NO:5), A6 (SEQ ID NO:6), A7 (SEQ ID NO:7), A8 (SEQ ID NO:8), and A9 (SEQ ID NO:9).

Embodiment 6 provides the composition of Embodiment 4 or 5, wherein the DNA oligonucleotide is selected from the group consisting of A1 (SEQ ID NO:1), A2 (SEQ ID NO:2), A3 (SEQ ID NO:3), A4 (SEQ ID NO:4), A5 (SEQ ID NO:5), A6 (SEQ ID NO:6), A7 (SEQ ID NO: 7), A8 (SEQ ID NO:8), and A9 (SEQ ID NO:9).

Embodiment 7 provides the composition of Embodiment 3, wherein the aptamer comprises a RNA oligonucleotide.

Embodiment 8 provides the composition of Embodiment 7, wherein the RNA oligonucleotide shares at least 85% 90%, 95%, 96%, 97%, 98%, or 99% sequence homology with a RNA oligonucleotide selected from the group consisting of A10 (SEQ ID NO: 10) and A11 (SEQ ID NO:11).

Embodiment 9 provides the composition of Embodiment 7 or 8, wherein the RNA oligonucleotide is selected from the group consisting of A10 (SEQ ID NO:10) and A11 (SEQ ID NO: 11).

Embodiment 10 provides the composition of any one of Embodiments 3-9, wherein the solid support is selected from the group consisting of controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, polyvinyl butyrate, agarose, and sepharose.

Embodiment 11 provides the composition of any one of Embodiments 3-10, wherein the solid support has a spherical morphology (e.g., a bead).

Embodiment 12 provides the composition of Embodiment 11, wherein the bead has a diameter ranging from about 1 nm to about 500 nm, optionally wherein the bead has a diameter ranging from about 1 nm to about 100 nm (i.e., a nanoparticle).

Embodiment 13 provides the composition of Embodiment 11, wherein the bead has a diameter ranging from about 1 μm to about 1000 μm (i.e., a microparticle).

Embodiment 14 provides the composition of any one of Embodiments 11-13, wherein the bead is magnetic.

Embodiment 15 provides the composition of any one of Embodiments 3-14, wherein the aptamer is conjugated to the solid support via at least one bond or bonding interaction selected from the group consisting of a covalent bond, an ionic bond, a hydrogen bond, and a Van der Waals bonding interaction.

Embodiment 16 provides the composition of any one of Embodiments 3-15, wherein the aptamer is conjugated to the solid support via a cross-linker.

Embodiment 17 provides the composition of Embodiment 16, wherein the cross-linker comprises polyethylenimine (PEI).

Embodiment 18 provides the composition of Embodiment 2, wherein the histamine binding domain is an anti-histamine antibody.

Embodiment 19 provides the composition of Embodiment 18, wherein the anti-histamine antibody is selected from the group consisting of a polyclonal antibody, a monoclonal antibody, a synthetic antibody, a human antibody, a humanized antibody, a non-human antibody, and a single-chain variable fragment (scFy) antibody, a single-domain antibody (sdAb), or any fragment thereof.

Embodiment 20 provides the composition of Embodiment 19, wherein the anti-histamine antibody is a polyclonal antibody.

Embodiment 21 provides the composition of Embodiment 20, wherein the anti-histamine antibody is produced in at least one animal selected from the group consisting of a mouse, rat, hamster, guinea pig, chicken, rabbit, donkey, llama, camel, goat, sheep, horse, and cow.

Embodiment 22 provides the composition of any one of Embodiments 18-21, wherein the solid support is selected from the group consisting of sepharose, agarose, controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, and polyvinyl butyrate.

Embodiment 23 provides the composition of Embodiment 22, wherein the sepharose comprises Protein A sepharose or Protein sepharose G.

Embodiment 24 provides the composition of Embodiment 22 or 23, wherein the Protein A and the histamine binding domain have a molar ratio of about 1:1 to about 1:10.

Embodiment 25 provides the composition of any one of Embodiments 18-24, wherein the histamine binding domain is conjugated to the solid support via a cross-linker.

Embodiment 26 provides the composition of Embodiment 25, wherein the cross-linker comprises suberic acid bis(N-hydroxysuccinimide).

Embodiment 27 provides the composition of any one of Embodiments 18-26, wherein the solid support has a spherical morphology (e.g., a bead).

Embodiment 28 provides the composition of Embodiment 27, wherein the bead has a diameter ranging from about 1 nm to about 500 nm, optionally wherein the bead has a diameter ranging from about 1 nm to about 100 nm.

Embodiment 29 provides the composition of Embodiment 27, wherein the bead has a diameter ranging from about 1 μm to about 1000 μm, optionally wherein the bead has a diameter ranging from about 40 μm to about 170 μm.

Embodiment 30 provides the composition of Embodiment 27 or 29, wherein the bead has an average diameter of about 90 μm.

Embodiment 31 provides the composition of any one of Embodiments 27-30, wherein the bead is magnetic.

Embodiment 32 provides the composition of any one of Embodiments 27-31, wherein the bead is non-porous.

Embodiment 33 provides a histamine scavenging apparatus comprising a porous container having a histamine-scavenging agent held therein, wherein the porous container comprises a plurality of pores having a diameter of sufficient size such that:
(a) passage of the histamine scavenging agent and optionally one or more particles within the liquid medium therethrough is prevented; and
(b) passage of a liquid medium therethrough is permitted.

Embodiment 34 provides the apparatus of Embodiment 33, wherein the histamine scavenging agent comprises the composition of any one of Embodiments 1-32.

Embodiment 35 provides the apparatus of Embodiment 33 or 34, wherein the porous container comprises nylon.

Embodiment 36 provides the apparatus of any one of Embodiments 33-35, wherein the pore diameter is about 25 μm.

Embodiment 37 provides the apparatus of any one of Embodiments 33-36, wherein the porous container comprises a tea bag.

Embodiment 38 provides the apparatus of Embodiment 37, wherein the tea bag is sealed with sealing wax.

Embodiment 39 provides a histamine scavenging apparatus comprising a column having a histamine scavenging agent held therein, an inlet for a liquid medium comprising histamine, and an outlet for a treated liquid medium with reduced histamine content, wherein the outlet comprises a porous and/or permeable material of sufficient porosity and/or permeability such that:
(a) passage of the histamine scavenging agent therethrough is prevented; and
(b) passage of the treated liquid medium therethrough is permitted.

Embodiment 40 provides the apparatus of Embodiment 39, wherein the histamine scavenging agent comprises the composition of any one of Embodiments 1-32.

Embodiment 41 provides the apparatus of Embodiment 39 or 40, wherein the liquid medium is a fermented liquid for human consumption.

Embodiment 42 provides the apparatus of any one of Embodiments 39-41, wherein the liquid medium is selected from the group consisting of wine, beer, cider, mead, sake, vinegar, soy sauce, tepache, and kombucha.

Embodiment 43 provides the apparatus of any one of Embodiments 39-42, wherein the fermented liquid with reduced histamine content has a histamine concentration of less than about 0.01 mg/L to about 25.00 mg/L.

Embodiment 44 provides the apparatus of any one of Embodiments 39-43, wherein the fermented liquid with reduced histamine content has a histamine concentration which is at least 10% lower than the histamine concentration of the liquid medium comprising histamine.

Embodiment 45 provides a method of treating a fermented liquid comprising histamine, the method comprising contacting the fermented liquid with a histamine scavenging agent to provide a fermented liquid with reduced histamine content and a histamine adsorbed species.

Embodiment 46 provides the method of Embodiment 45, wherein the histamine scavenging agent comprises the composition of any one of Embodiments 1-32.

Embodiment 47 provides the method of Embodiment 45 or 46, wherein the contacting occurs by immersion of the histamine scavenging agent in the fermented liquid.

Embodiment 48 provides the method of Embodiment 47, wherein the immersion occurs for a period of about 1 min to about 60 min.

Embodiment 49 provides the method of any one of Embodiments 45-48, wherein the contacting occurs with agitation.

Embodiment 50 provides the method of Embodiment 45 or 46, wherein the contacting occurs by passage of the fermented liquid through a stationary phase comprising the histamine scavenging agent.

Embodiment 51 provides the method of any one of Embodiments 45-50, wherein the fermented liquid is alcoholic or non-alcoholic.

Embodiment 52 provides the method of any one of Embodiments 45-51, wherein the fermented liquid is a fermented liquid beverage for human consumption.

Embodiment 53 provides the method of any one of Embodiments 45-52, wherein the fermented liquid is selected from the group consisting of wine, beer, cider, mead, sake, vinegar, soy sauce, tepache, and kombucha.

Embodiment 54 provides the method of any one of Embodiments 45-53, wherein the fermented liquid with reduced histamine content has a histamine concentration of less than about 0.01 mg/L to about 25.00 mg/L.

Embodiment 55 provides the method of any one of Embodiments 45-54, wherein the fermented liquid with reduced histamine content has a histamine concentration which is at least 10% lower than the histamine concentration of the liquid medium comprising histamine.

Embodiment 56 provides the method of any one of Embodiments 45-55, further comprising separating the fermented liquid with reduced histamine content and the histamine adsorbed species.

Embodiment 57 provides the method of Embodiment 56, wherein the histamine adsorbed species comprises a magnetic solid support, optionally wherein the solid support has a spherical morphology (e.g., a bead), and optionally wherein the bead comprises steel.

Embodiment 58 provides the method of Embodiment 57, wherein the histamine adsorbed species is separated from the fermented liquid with reduced histamine content by magnetic separation.

Embodiment 59 provides the method of any one of Embodiments 45-58, further comprising regenerating the histamine scavenging agent, comprising the steps of:
(a) contacting the histamine adsorbed species with a low pH solution or a denaturant to provide a first washed species; and
(b) contacting the first washed species with a buffered solution to provide the histamine scavenging agent.

Embodiment 60 provides the method of Embodiment 59, wherein the low pH solution comprises a glycine solution.

Embodiment 61 provides the method of Embodiment 60, wherein the glycine solution has a glycine concentration of about 0.05 M to about 0.5 M.

Embodiment 62 provides the method of any one of Embodiments 59-61, wherein the low pH solution has a pH of about 2.2.

Embodiment 63 provides the method of Embodiment 59, wherein the denaturant comprises a urea solution.

Embodiment 64 provides the method of Embodiment 63, wherein the urea solution has a urea concentration ranging from about 1 M to about 8 M.

Embodiment 65 provides the method of any one of Embodiments 59-64, wherein the buffered solution comprises a phosphate-buffered saline (PBS) solution or ethylenediaminetetraacetic acid (EDTA) solution.

Embodiment 66 provides the method of any one of Embodiments 59-65, wherein the histamine scavenging agent is regenerated without a loss in histamine scavenging efficiency.

Embodiment 67 provides the method of Embodiment 66, wherein the histamine scavenging agent is stored in a suspension buffer solution after regeneration.

Embodiment 68 provides the method of Embodiment 67, wherein the suspension buffer solution comprises a 20% EtOH phosphate-buffered saline (PBS) solution or ethylenediaminetetraacetic acid (EDTA) solution.

Embodiment 69 provides the method of Embodiment 67, wherein the histamine scavenging agent can be stored long term in a 20% EtOH phosphate-buffered saline (PBS) solution or ethylenediaminetetraacetic acid (EDTA) solution.

Embodiment 70 provides the method of any one of Embodiments 65-69, wherein the histamine scavenging agent is washed with the buffered solution prior to a subsequent use in histamine removal from a second fermented liquid.

Embodiment 71 provides a kit comprising the composition of any one of Embodiments 1-32, and instructional material for use thereof, wherein the instructional material comprises instructions for at least partially removing histamine from a fermented liquid.

Embodiment 72 provides a kit comprising a parcel containing the histamine scavenging composition of any one of Embodiments 1-32, a low pH solution or a denaturant, a wash buffer solution, and a suspension buffer solution.

Embodiment 73 provides the kit of Embodiment 72, wherein the low pH solution comprises a glycine solution.

Embodiment 74 provides the kit of Embodiment 73, wherein the glycine solution has a glycine concentration of about 0.05 M to about 0.5 M.

Embodiment 75 provides the kit of any one of Embodiments 72-74, wherein the low pH solution has a pH of about 2.2.

Embodiment 76 provides the kit of Embodiment 72, wherein the denaturant comprises a urea solution.

Embodiment 77 provides the kit of Embodiment 76, wherein the urea solution has a urea concentration ranging from about 1 M to about 8 M.

Embodiment 78 provides the kit of any one of Embodiments 72-77, wherein the wash buffer solution comprises a phosphate-buffered saline (PBS) solution or ethylenediaminetetraacetic acid (EDTA) solution.

Embodiment 79 provides the kit of any one of Embodiments 72-78, wherein the suspension buffer solution comprises a 20% EtOH phosphate-buffered saline (PBS) solution or ethylenediaminetetraacetic acid (EDTA) solution.

Embodiment 80 provides a method of at least partially removing histamine from a fermented liquid using the kit of any one of Embodiments 72-79 to provide a fermented liquid with reduced histamine content, the method comprising:
contacting the parcel comprising the histamine scavenging composition with the fermented liquid to provide a fermented liquid with reduced histamine content and a parcel comprising a histamine adsorbed species;
separating the fermented liquid with reduced histamine content and the parcel comprising the histamine adsorbed species;
contacting the parcel comprising the histamine adsorbed species with the low pH solution or denaturant to provide a parcel comprising a first washed species;
contacting the parcel comprising the first washed species with the wash buffer solution to regenerate the parcel comprising the histamine scavenging composition; and
optionally storing the parcel comprising the histamine scavenging composition in the stored suspension buffer.

Embodiment 81 provides the method of Embodiment 80, wherein the regenerated parcel comprising the histamine scavenging composition is used to at least partially remove histamine from a second fermented liquid.

Embodiment 82 provides the method of Embodiment 81, wherein the parcel comprising the histamine scavenging composition is separated from the suspension buffer and contacted with the wash buffer solution.

Embodiment 83 provides the method of any one of Embodiments 80-82, wherein the fermented liquid is a fermented liquid beverage for human consumption.

Embodiment 84 provides the method of Embodiment 83, wherein the beverage for human consumption is selected from the group consisting of wine, beer, cider, mead, sake, vinegar, soy sauce, tepache, and kombucha.

Embodiment 85 provides the method of Embodiment 84, wherein the wine is red wine.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A composition comprising a histamine binding domain conjugated to a solid support.

2. The composition of claim 1, wherein the histamine binding domain is selected from the group consisting of an aptamer, a histamine binding fragment (Fab), a single-chain variable fragment (scFv), a single-domain antibody (sdAb), an anti-histamine antibody, and an anticalin.

3. (canceled)

4. The composition of claim 2, wherein at least one of the following applies:
(a) the aptamer comprises a DNA oligonucleotide, optionally wherein the DNA oligonucleotide shares at least 85%, 90%, 95%, 96%, 97%, 98%, or 99% sequence homology with a DNA oligonucleotide selected from the group consisting of A1 (SEQ ID NO:1), A2 (SEQ ID NO: 2), A3 (SEQ ID NO:3), A4 (SEQ ID NO:4), A5 (SEQ ID NO:5), A6 (SEQ ID NO:6), A7 (SEQ ID NO:7), A8 (SEQ ID NO:8), and A9 (SEQ ID NO:9); and
(b) the aptamer comprises a RNA oligonucleotide, optionally wherein the RNA oligonucleotide shares at least 85% 90%, 95%, 96%, 97%, 98%, or 99% sequence homology with a RNA oligonucleotide selected from the group consisting of A10 (SEQ ID NO: 10) and A11 (SEQ ID NO:11).

5-9. (canceled)

10. The composition of claim 2, wherein at least one of the following applies:
(a) the solid support is selected from the group consisting of controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, polyvinyl butyrate, agarose, and sepharose;
(b) the solid support has a spherical (bead) morphology, optionally wherein the bead has a diameter ranging from about 1 nm to about 500 nm (nanoparticle) or the bead has a diameter ranging from about 1 μm to about 1000 μm (microparticle), optionally wherein the bead is magnetic, and optionally wherein the bead is non-porous;
(c) the aptamer is conjugated to the solid support via at least one bond or bonding interaction selected from the group consisting of a covalent bond, an ionic bond, a hydrogen bond, and a Van der Waals bonding interaction; and
(d) the aptamer is conjugated to the solid support via a cross-linker, optionally wherein the cross-linker comprises polyethylenimine (PEI) or suberic acid bis(N-hydroxysuccinimide).

11-18. (canceled)

19. The composition of claim 2, wherein at least one of the following applies:
(a) the anti-histamine antibody is selected from the group consisting of a polyclonal antibody, a monoclonal antibody, a synthetic antibody, a human antibody, a humanized antibody, a non-human antibody, and a single-chain variable fragment (scFy) antibody, a single-domain antibody (sdAb), or any fragment thereof;
(b) the anti-histamine antibody is a polyclonal antibody; and
(c) the anti-histamine antibody is produced in at least one animal selected from the group consisting of a mouse, rat, hamster, guinea pig, chicken, rabbit, donkey, llama, camel, goat, sheep, horse, and cow.

20-21. (canceled)

22. The composition of claim 19, wherein the solid support is selected from the group consisting of sepharose, agarose, controlled pore glass (CPG), silica gel, polystyrene, polyvinyl chloride, polyacrylamide, polyacrylate, polyethylene, polypropylene, poly(4-methylbutene), polystyrene, polymethacrylate, poly(ethylene terephthalate), polytetrafluoroethylene (PTFE), steel, nylon, and polyvinyl butyrate, optionally wherein the sepharose comprises Protein sepharose A or Protein Sepharose G, and optionally wherein the Protein A and the histamine binding domain have a molar ratio of about 1:1 to about 1:10.

23-26. (canceled)

27. The composition of claim 19, wherein the solid support has a spherical (bead) morphology and one of the following applies:
(a) the bead has a diameter ranging from about 1 nm to about 500 nm, optionally wherein the bead has a diameter ranging from about 1 nm to about 100 nm; and
(b) the bead has a diameter ranging from about 1 μm to about 1000 μm, optionally wherein the bead has a diameter ranging from about 40 μm to about 170 μm, optionally wherein the bead has an average diameter of about 90 μm.

28-32. (canceled)

33. A histamine scavenging apparatus comprising a porous container having a histamine-scavenging agent held therein, wherein the porous container comprises a plurality of pores having a diameter of sufficient size such that:
(a) passage of the histamine scavenging agent and optionally one or more particles within the liquid medium therethrough is prevented; and
(b) passage of a liquid medium therethrough is permitted.

34. The apparatus of claim 33, wherein at least one of the following applies:
(a) the histamine scavenging agent comprises a composition comprising a histamine binding domain conjugated to a solid support;
(b) the porous container comprises nylon;
(c) the pore diameter is about 25 μm; and
(d) the porous container comprises a tea bag, optionally wherein the tea bag is sealed with sealing wax.

35-38. (canceled)

39. A histamine scavenging apparatus comprising a column having a histamine scavenging agent held therein, an inlet for a liquid medium comprising histamine, and an outlet for a treated liquid medium with reduced histamine content, wherein the outlet comprises a porous and/or permeable material of sufficient porosity and/or permeability such that:
(a) passage of the histamine scavenging agent therethrough is prevented; and
(b) passage of the treated liquid medium therethrough is permitted.

40. The apparatus of claim 39, wherein at least one of the following applies:
(a) the histamine scavenging agent comprises a composition comprising a histamine binding domain conjugated to a solid support;
(b) the liquid medium is a fermented liquid for human consumption;
(c) the liquid medium is selected from the group consisting of wine, beer, cider, mead, sake, vinegar, soy sauce, tepache, and kombucha;
(d) the fermented liquid with reduced histamine content has a histamine concentration of less than about 0.01 mg/L to about 25.00 mg/L; and
(e) the fermented liquid with reduced histamine content has a histamine concentration which is at least 10% lower than the histamine concentration of the liquid medium comprising histamine.

41-44. (canceled)

45. A method of treating a fermented liquid comprising histamine, the method comprising contacting the fermented liquid with a histamine scavenging agent to provide a fermented liquid with reduced histamine content and a histamine adsorbed species, wherein the histamine scavenging agent comprises the composition of claim 1.

46. The method of claim 45, wherein at least one of the following applies:
(a) the contacting occurs by immersion of the histamine scavenging agent in the fermented liquid;
(b) the immersion occurs for a period of about 1 min to about 60 min;
(c) the contacting occurs with agitation;
(d) the contacting occurs by passage of the fermented liquid through a stationary phase comprising the histamine scavenging agent;
(e) the fermented liquid is alcoholic or non-alcoholic;
(f) the fermented liquid is a fermented liquid beverage for human consumption;
(g) the fermented liquid is selected from the group consisting of wine, beer, cider, mead, sake, vinegar, soy sauce, tepache, and kombucha;
(h) the fermented liquid with reduced histamine content has a histamine concentration of less than about 0.01 mg/L to about 25.00 mg/L; and
(i) the fermented liquid with reduced histamine content has a histamine concentration which is at least 10% lower than the histamine concentration of the liquid medium comprising histamine.

47-55. (canceled)

56. The method of claim 45, further comprising separating the fermented liquid with reduced histamine content and the histamine adsorbed species, optionally wherein the histamine adsorbed species comprises a magnetic solid support, optionally wherein the solid support has a spherical (bead) morphology, optionally wherein the bead comprises steel, and optionally wherein the histamine adsorbed species is separated from the fermented liquid with reduced histamine content by magnetic separation.

57-58. (canceled)

59. The method of claim 45, further comprising regenerating the histamine scavenging agent, comprising the steps of:
(a) contacting the histamine adsorbed species with a low pH solution or a denaturant to provide a first washed species; and
(b) contacting the first washed species with a buffered solution to provide the histamine scavenging agent.

60. The method of claim 59, wherein at least one of the following applies:
(a) the low pH solution comprises a glycine solution, optionally wherein the glycine solution has a glycine concentration of about 0.05 M to about 0.5 M;
(b) the low pH solution has a pH of about 2.2;
(c) the denaturant comprises a urea solution, optionally wherein the urea solution has a urea concentration ranging from about 1 M to about 8 M;
(d) the buffered solution comprises a phosphate-buffered saline (PBS) solution or ethylenediaminetetraacetic acid (EDTA) solution;
(e) the histamine scavenging agent is regenerated without a loss in histamine scavenging efficiency;
(f) the histamine scavenging agent is stored in a suspension buffer solution after regeneration, optionally wherein the suspension buffer solution comprises a 20% EtOH phosphate-buffered saline (PBS) solution or ethylenediaminetetraacetic acid (EDTA) solution, and optionally wherein the histamine scavenging agent is washed with the buffered solution prior to a subsequent use in histamine removal from a second fermented liquid; and
(g) the histamine scavenging agent can be stored long term in a 20% EtOH phosphate-buffered saline (PBS) solution or ethylenediaminetetraacetic acid (EDTA) solution.

61-70. (canceled)

71. A kit comprising the composition of claim 1, and instructional material for use thereof, wherein the instructional material comprises instructions for at least partially removing histamine from a fermented liquid.

72. A kit comprising a parcel containing the composition of claim 1, a low pH solution or a denaturant, a wash buffer solution, and a suspension buffer solution.

73-79. (canceled)

80. A method of at least partially removing histamine from a fermented liquid using the kit of claim 72 to provide a fermented liquid with reduced histamine content, the method comprising:
contacting the parcel comprising the histamine scavenging composition with the fermented liquid to provide a fermented liquid with reduced histamine content and a parcel comprising a histamine adsorbed species;
separating the fermented liquid with reduced histamine content and the parcel comprising the histamine adsorbed species;
contacting the parcel comprising the histamine adsorbed species with the low pH solution or denaturant to provide a parcel comprising a first washed species;
contacting the parcel comprising the first washed species with the wash buffer solution to regenerate the parcel comprising the histamine scavenging composition; and
optionally storing the parcel comprising the histamine scavenging composition in the stored suspension buffer.

81. The method of claim 80, wherein at least one of the following applies:
(a) the regenerated parcel comprising the histamine scavenging composition is used to at least partially remove histamine from a second fermented liquid;
(b) the parcel comprising the histamine scavenging composition is separated from the suspension buffer and contacted with the wash buffer solution;
(c) the fermented liquid is a fermented liquid beverage for human consumption;
(d) the beverage for human consumption is selected from the group consisting of wine beer, cider, mead, sake, vinegar, soy sauce tepache, and kombucha; and
(e) the wine is red wine.

82-85. (canceled)
